(19) United States
(12) Patent Application Publication
(10) Pub. No.: US 2024/0215987 A1
(43) Pub. Date: Jul. 4, 2024

(54) ATTACHMENT DEVICE AND ATTACHMENT METHOD

(71) Applicant: TERUMO KABUSHIKI KAISHA, Tokyo (JP)

(72) Inventor: Masakatsu KAWAURA, Hiratsuka city (JP)

(73) Assignee: TERUMO KABUSHIKI KAISHA, Tokyo (JP)

(21) Appl. No.: 18/598,809

(22) Filed: Mar. 7, 2024

Related U.S. Application Data
(63) Continuation of application No. PCT/JP2022/033621, filed on Sep. 7, 2022.

(30) Foreign Application Priority Data
Sep. 29, 2021 (JP) 2021-159817

(51) Int. Cl. A61B 17/132; A61B 17/00; A61B 17/12; A61B 17/135

(52) U.S. Cl. CPC A61B 17/1325; A61B 17/135; A61B 2017/00951; A61B 2017/12004

(57) ABSTRACT

An attachment device according to the present disclosure includes an attachment sheet including an attachment surface attachable to a living body surface; a device body fixed to the attachment sheet; and a plurality of release sheets attached to the attachment surface, in which the attachment sheet includes an outer edge portion to which the device body is not fixed and that is positioned at an outer periphery of the device body in plan view as viewed in a thickness direction of the attachment sheet, and the plurality of release sheets include a first release sheet that is attached to a first attachment region including an attachment surface of a part different from the outer edge portion in the attachment surface, and a second release sheet that is attached to a second attachment region including only an attachment surface of the outer edge portion in the attachment surface.

### CROSS-REFERENCES TO RELATED APPLICATIONS

This application is a continuation of International Application No. PCT/JP2022/033621 filed on Sep. 7, 2022, which claims priority to Japanese Application No. 2021-159817 filed on Sep. 29, 2021, the entire content of both of which is incorporated herein by reference.

### TECHNOLOGICAL FIELD

The present disclosure generally relates to an attachment device and an attachment method.

### BACKGROUND DISCUSSION

In recent years, in medical institutions, various forms of examinations or treatments have been performed using an elongated hollow tubular insertion member for medical use called a catheter. The catheter is percutaneously inserted into a blood vessel from a puncture site formed on the wrist, groin, or the like, and is carried to a site to be examined or treated through the blood vessel. After completion of the examination or treatment by a medical worker, the catheter and the sheath used for catheter introduction are removed from the puncture site, and hemostasis of bleeding from the puncture site is performed.

International Patent Application Publication No. WO 2020/050420 A discloses an attachment device including a pressing member capable of pressing a puncture site on a living body surface. In addition, the attachment device in International Patent Application Publication No. WO 2020/050420 A includes a release sheet that is releasably attached to an attachment surface of an attachment sheet.

According to the attachment device in International Patent Application Publication No. WO 2020/050420 A, it is possible to realize a configuration that is unlikely to peel from a living body surface. In addition, according to the attachment device in International Patent Application Publication No. WO 2020/050420 A, it is possible to realize a release sheet that is easily released from an attachment surface of an attachment sheet. However, the attachment device in International Patent Application Publication No. WO 2020/050420 A still has room for improvement from the viewpoint of improving the ease of releasing the release sheet from the attachment surface of the attachment sheet.

### SUMMARY

An attachment device and an attachment method in which a release sheet is easily released from an attachment surface of an attachment sheet, and the attachment surface of the attachment sheet is unlikely to peel from a living body surface.

As a first aspect of the present disclosure, provided is an attachment device including: an attachment sheet including an attachment surface configured to be attached to a living body surface and a fixing surface that is positioned on a side opposite to the attachment surface; a device body that is fixed to the fixing surface of the attachment sheet; and a plurality of release sheets that are releasably attached to different positions on the attachment surface of the attachment sheet, in which the attachment sheet includes an outer edge portion to which the device body is not fixed and that is positioned at an outer periphery of the device body in plan view as viewed in a thickness direction of the attachment sheet, and the plurality of release sheets include a first release sheet that is attached to a first attachment region including an attachment surface of a part different from the outer edge portion in the attachment surface of the attachment sheet, and a second release sheet that is attached to a second attachment region including only an attachment surface of the outer edge portion in the attachment surface of the attachment sheet.

As one embodiment of the present disclosure, the first release sheet includes a first release layer that is attached to the first attachment region of the attachment surface of the attachment sheet, and a first folding layer that is folded and laminated on the first release layer on a side of the attachment surface opposite to a first attachment region side.

As one embodiment of the present disclosure, the second release sheet includes a second release layer that is attached to the second attachment region of the attachment surface of the attachment sheet, and a second folding layer that is folded and laminated on the second release layer on a side of the attachment surface opposite to a second attachment region side.

As one embodiment of the present disclosure, a first folding direction of the first release sheet from the first release layer to the first folding layer is different from a second folding direction of the second release sheet from the second release layer to the second folding layer.

As one embodiment of the present disclosure, the first folding direction and the second folding direction are in mutually opposite directions in an in-plane direction of the attachment surface of the attachment sheet.

As one embodiment of the present disclosure, the second attachment region is adjacent to the first attachment region.

As one embodiment of the present disclosure, the first release sheet includes an overlapping portion that covers the second release sheet.

As one embodiment of the present disclosure, a boundary between the first attachment region and the second attachment region is defined by a ridgeline formed by bending of the outer edge portion of the attachment sheet toward a fixing surface side, and the overlapping portion of the first release sheet protrudes in a direction away from the second release sheet along the first attachment region, not along the second attachment region.

As one embodiment of the present disclosure, a boundary between the first attachment region and the second attachment region is defined by a ridgeline formed by bending of the outer edge portion of the attachment sheet toward a fixing surface side, and the overlapping portion of the first release sheet extends along the second attachment region and covers the second release sheet.

As one embodiment of the present disclosure, the device body includes a locking portion that locks the second release sheet in a state in which the outer edge portion of the attachment sheet is bent toward the fixing surface side.

As one embodiment of the present disclosure, the locking portion is a recess that an outer edge portion of the second release sheet enters.

As a second aspect of the present disclosure, an attachment device comprising: an attachment sheet including an attachment surface configured to be attached to a living body surface and a fixing surface that is positioned on a side opposite to the attachment surface, the fixing surface configured to receive a device body including a pressing portion configured to press the living body surface in a central region of the attachment sheet; a plurality of release sheets that are releasably attached to different positions on the attachment surface of the attachment sheet; and wherein the plurality of release sheets include a first release sheet that is attached to a first attachment region including an attachment surface of a part different from an outer edge portion in the attachment surface of the attachment sheet, and a second release sheet that is attached to a second attachment region including only an attachment surface of the outer edge portion in the attachment surface of the attachment sheet.

As a third aspect of the present disclosure, provided is an attachment method for attachment to a living body surface by an attachment device, the attachment device including an attachment sheet including an attachment surface that is attachable to a living body surface and a fixing surface that is positioned on a side opposite to the attachment surface, a device body that is fixed to the fixing surface of the attachment sheet, and first and second release sheets that are releasably attached to different positions on the attachment surface of the attachment sheet, the first release sheet being attached to a first attachment region in the attachment surface of the attachment sheet, and the second release sheet being attached to a second attachment region in the attachment surface of the attachment sheet, the method including: a first attachment step of releasing the first release sheet from the first attachment region, and attaching the first attachment region to the living body surface; and a second attachment step of releasing the second release sheet from the second attachment region by moving the second release sheet in a direction orthogonal to the first attachment region in a state attached to the living body surface, and attaching the second attachment region to the living body surface.

According to the present disclosure, it is possible to provide an attachment device and an attachment method in which a release sheet is easily released from an attachment surface of an attachment sheet, and the attachment surface of the attachment sheet is unlikely to peel from a living body surface.

### BRIEF DESCRIPTION OF THE DRAWINGS

FIG. 1 is a perspective view of an attachment device as one embodiment of the present disclosure.

FIG. 2 is an exploded perspective view of the attachment device shown in FIG. 1.

FIG. 3 is a top view of the attachment device shown in FIG. 1.

FIG. 4 is a bottom view of the attachment device shown in FIG. 1.

FIG. 5 is a side view of the attachment device shown in FIG. 1.

FIG. 6 is another side view of the attachment device shown in FIG. 1.

FIG. 7 is a view showing a first release sheet of the attachment device shown in FIG. 1, and is a view showing a method of forming the first release sheet.

FIG. 8 is a view showing a second release sheet of the attachment device shown in FIG. 1, and is a view showing a method of forming the second release sheet.

FIG. 9 is a perspective view showing a state in which the first release sheet and the second release sheet of the attachment device shown in FIG. 1 are removed.

FIG. 10 is a cross-sectional view of the attachment device taken along line I-I in FIG. 9.

FIG. 11 is a cross-sectional view taken along the same line as FIG. 10 in a state in which an expansion portion is expanded.

FIGS. 12A to 12E are explanatory views explaining a procedure for removing a first release sheet and a second release sheet of the attachment device shown in FIG. 1.

FIG. 13 is a flowchart showing an example of a living body pressing method using the attachment device shown in FIG. 1.

FIG. 14A is a view showing an outline of a first release sheet removing step in FIG. 13.

FIG. 14B is a view showing an outline of a first attachment step in FIG. 13.

FIG. 14C is a view showing an outline of a first pressing step in FIG. 13.

FIG. 14D is a view showing an outline of a removing step in FIG. 13.

FIG. 14E is a view showing an outline of a second pressing step in FIG. 13.

FIG. 14F is a view showing an outline of a second release sheet removing step in FIG. 13.

FIG. 14G is a view showing an outline of a second attachment step in FIG. 13.

FIG. 15 is a perspective view of an attachment device as one embodiment of the present disclosure.

FIGS. 16A to 16E are explanatory views explaining a procedure for removing a first release sheet and a second release sheet of the attachment device shown in FIG. 15.

FIG. 17 is a top view of the attachment device shown in FIG. 15.

FIG. 18A is a view showing a state in which an insertion member is inserted from a living body surface into a femoral vein through a connective tissue.

FIG. 18B is a view showing a state after removal of the insertion members from the state shown in FIG. 18A.

FIG. 19 is a view showing a state in which the perforation shown in FIG. 18B is narrowed or occluded by the attachment device shown in FIG. 1.

FIG. 20 is a front view of the state shown in FIG. 19 as viewed from the living body surface side.

FIG. 21 is an explanatory view explaining a case where an external force in a direction orthogonal to a living body surface acts on a device body in a state in which the attachment device shown in FIG. 1 is attached to the living body surface.

### DETAILED DESCRIPTION

Set forth below with reference to the accompanying drawings is a detailed description of embodiments of an attachment device and an attachment method. In the drawings, the same components are denoted by the same reference numerals.

### First Embodiment

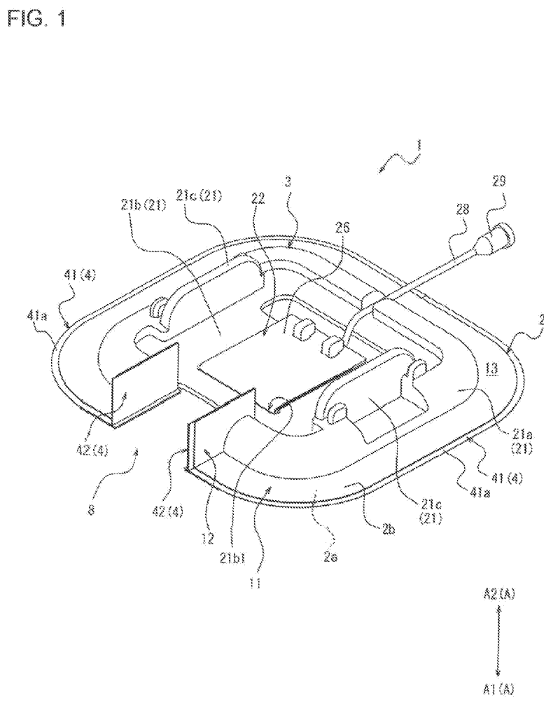

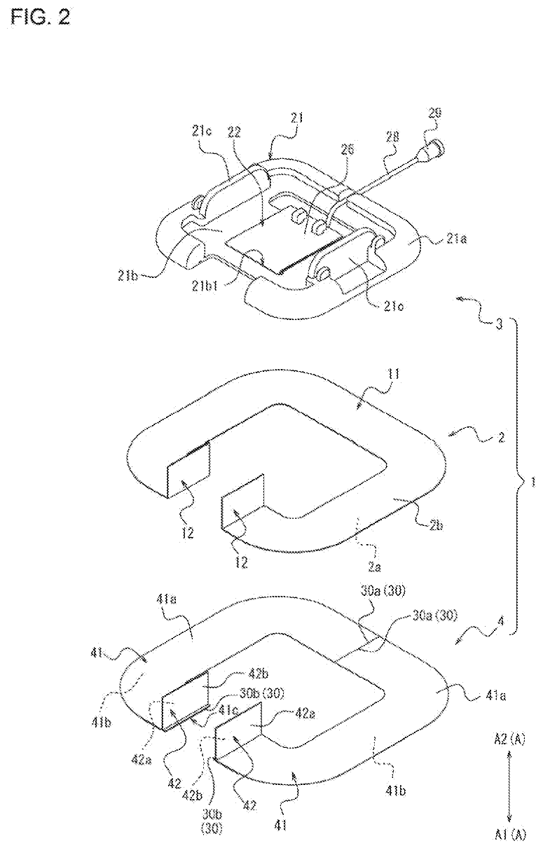

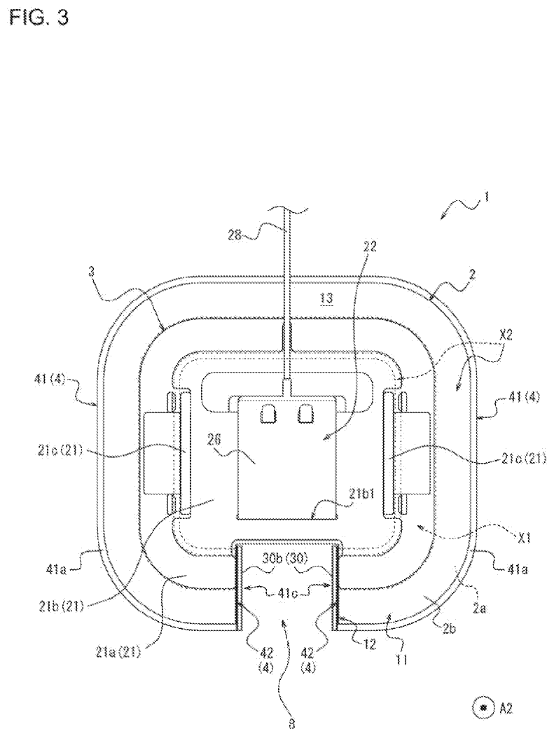

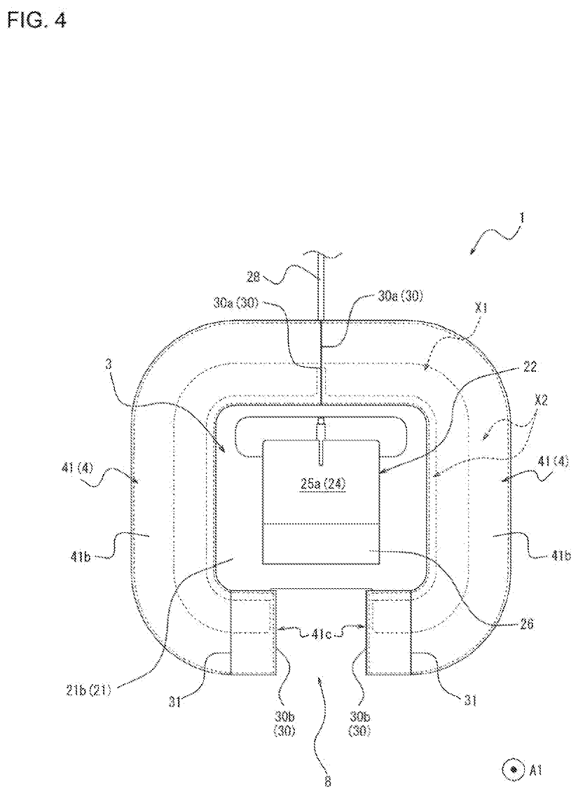

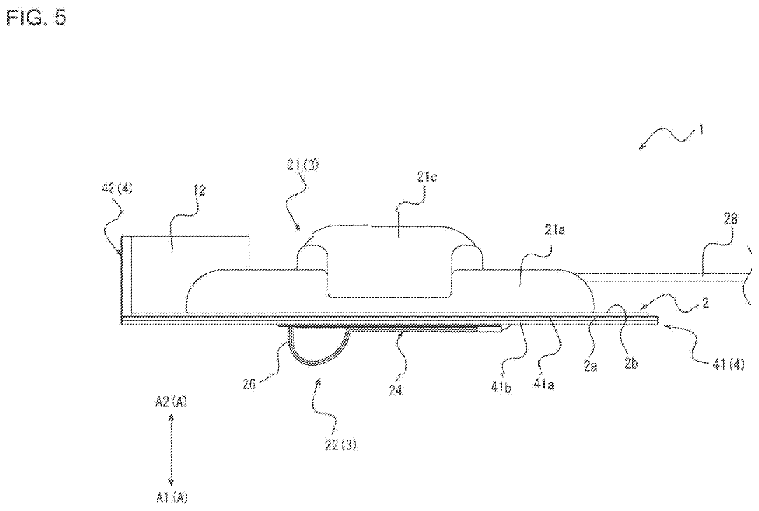

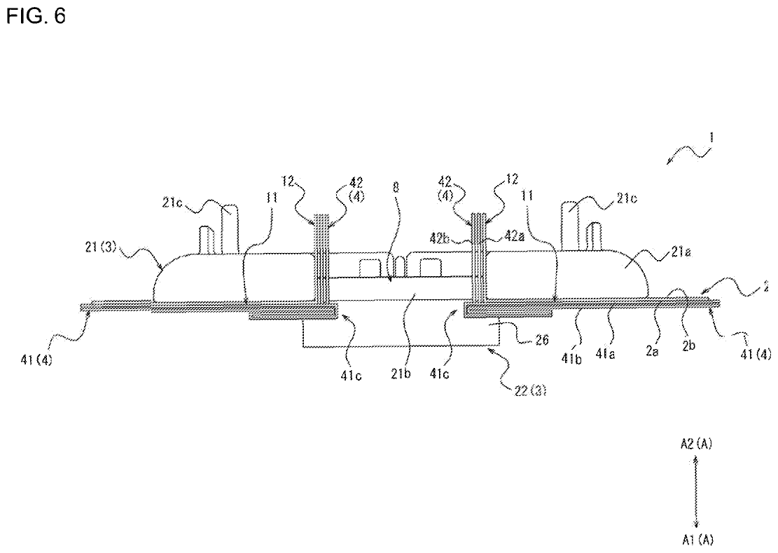

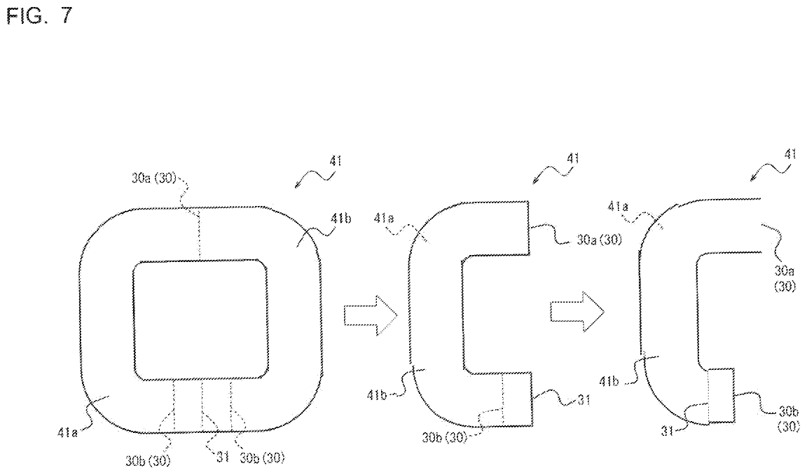

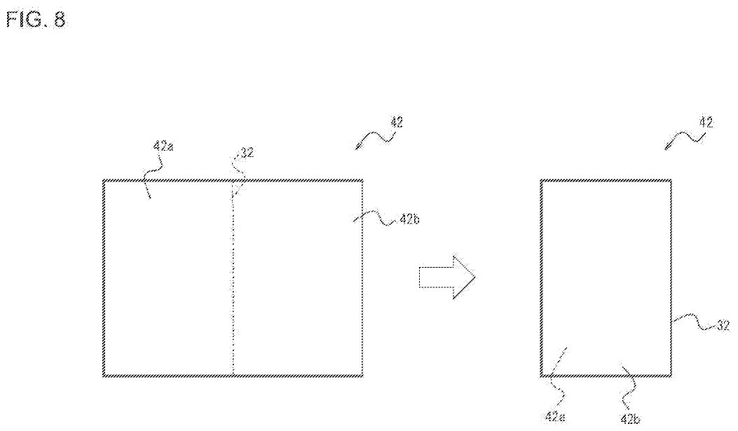

FIG. 1 is a perspective view of an attachment device 1 for medical use as a first embodiment of the present disclosure. FIG. 2 is an exploded perspective view of the attachment device 1. FIG. 3 is a top view of the attachment device 1. FIG. 4 is a bottom view of the attachment device 1. FIGS. 5 and 6 are side views of the attachment device 1 as viewed from different directions. FIG. 7 is a view showing a first release sheet 41 of the attachment device 1, and is a view showing a method of forming the first release sheet 41. FIG. 8 is a view showing a second release sheet 42 of the attachment device 1, and is a view showing a method of forming the second release sheet 42.

As shown in FIGS. 1 to 6, the attachment device 1 includes an attachment sheet 2, a device body 3, and a plurality of release sheets 4. The plurality of release sheets 4 include the first release sheet 41 and the second release sheet 42. In the present embodiment, in a case where the plurality of release sheets are not particularly distinguished, they are simply referred to as “release sheet 4.”

The attachment sheet 2 includes an attachment surface 2a that is attachable to a living body surface and a fixing surface 2b positioned on a side opposite to the attachment surface 2a. The attachment surface 2a is positioned on one side of the attachment sheet 2 in a thickness direction. The attachment surface 2b is positioned on the other side of the attachment sheet 2 in the thickness direction.

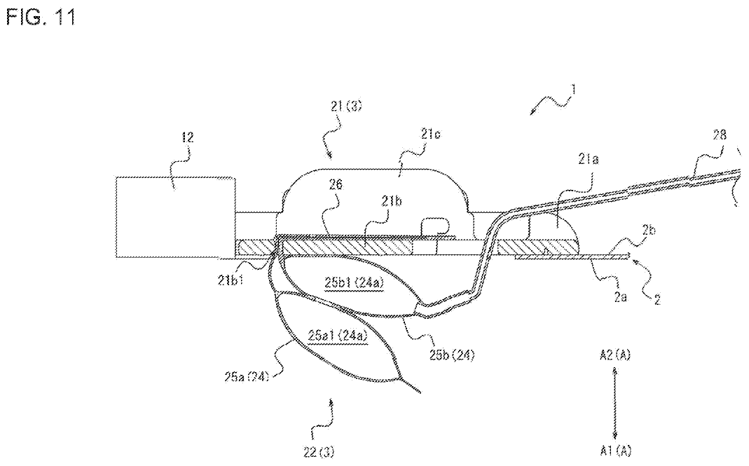

The device body 3 is fixed to the fixing surface 2b of the attachment sheet 2. The configuration of the device body 3 is not particularly limited. The device body 3 of the present embodiment can include a pressing body 22 (see FIG. 11, etc.) to be described later that is capable of pressing a living body surface, but is not limited to the configuration including the pressing body 22. The pressing body 22 will be described later in detail.

The release sheet 4 is releasably attached to the attachment surface 2a of the attachment sheet 2.

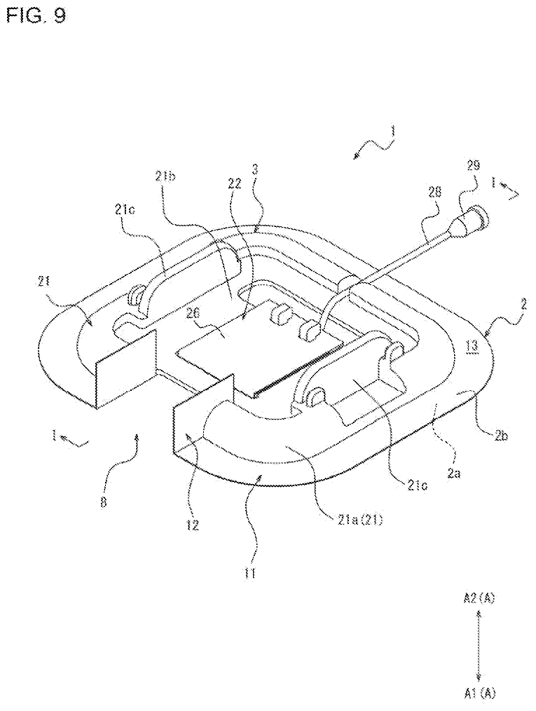

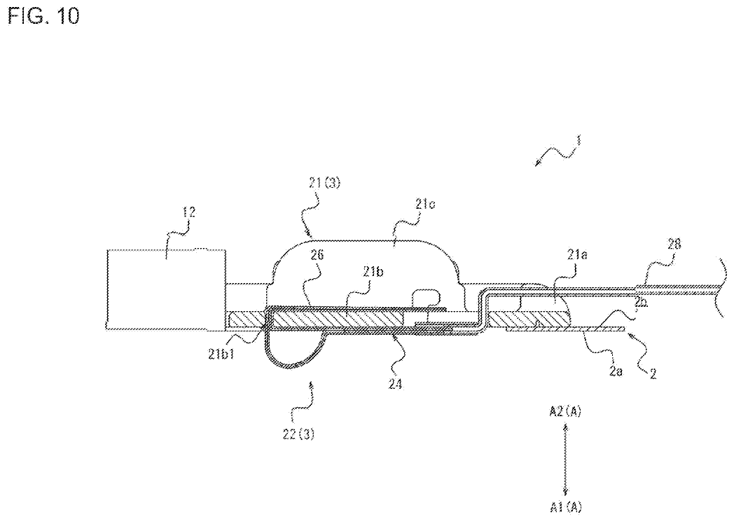

FIG. 9 is a perspective view showing a state in which all the release sheets 4 are released from the attachment device 1 shown in FIG. 1. FIGS. 10 and 11 are cross-sectional views of the attachment device 1 taken along line I-I in FIG. 9. FIG. 10 shows a case where an expansion portion 24 of the pressing body 22 to be described later is in a contracted state before expansion. In contrast, FIG. 11 shows a case where the expansion portion 24 of the pressing body 22 to be described later is in an expanded state after expansion. The position of the attachment device 1 on a living body surface is fixed by attaching the attachment surface 2a to the living body surface. The release sheet 4 is released from the attachment surface 2a when the attachment device 1 is attached to the living body surface. Therefore, the attachment surface 2a of the attachment sheet 2 is exposed to the outside, and the attachment device 1 can be attached to the living body surface.

Hereinafter, for explanatory convenience, a thickness direction A of the attachment sheet 2 in the attachment device 1 in a state in which the whole region of the attachment surface 2a is attached on a living body surface is simply referred to as “thickness direction A of the attachment sheet 2” or “thickness direction A.” Further, one side of the attachment sheet 2 in the thickness direction A that is in a direction from the fixing surface 2b toward the attachment surface 2a in the thickness direction A of the attachment sheet 2 is simply referred to as “downward direction A1” or “lower side.” In addition, for explanatory convenience, the other side of the attachment sheet 2 in the thickness direction A that is in a direction from the attachment surface 2a toward the fixing surface 2b in the thickness direction A of the attachment sheet 2 is simply referred to as “upward direction A2” or “upper side.” Therefore, the attachment surface 2a of the attachment sheet 2 of the present embodiment is constituted by the lower surface of the attachment sheet 2. In addition, the fixing surface 2b of the attachment sheet 2 of the present embodiment is constituted by the upper surface of the attachment sheet 2.

Hereinafter, among plan views (see FIGS. 3 and 4) of the attachment device 1 as viewed in the thickness direction A, a plan view (see FIG. 3) of the attachment device 1 as viewed from above in the downward direction A1 is simply referred to as “top view” for explanatory convenience. In addition, among the plan views (see FIGS. 3 and 4) of the attachment device 1 as viewed in the thickness direction A, a plan view (see FIG. 4) of the attachment device 1 as viewed from below in the upward direction A2 is simply referred to as “bottom view” for explanatory convenience. In addition, in a case where the top view and the bottom view are not particularly distinguished, they are simply referred to as “plan view.” In addition, unless otherwise specified, the terms “plan view,” “top view,” and “bottom view” simply refer to the plan view, top view, and bottom view in a case where the expansion portion 24 of the pressing body 22 to be described later is in a contracted state.

As shown in FIGS. 3 and 4, the attachment sheet 2 includes a first part X1 to which the device body 3 is fixed and a second part X2 to which the device body 3 is not fixed. The “first part X1” means a part of the attachment sheet 2 that overlaps the device body 3 in the thickness direction A and cannot be separated from the device body 3 in the thickness direction A. In addition, the “second part X2” means that it includes both (i) a part of the attachment sheet 2 that does not overlap the device body 3 in the thickness direction A and (ii) a part of the attachment sheet 2 that overlaps the device body 3 in the thickness direction A and can be separated from the device body 3 in the thickness direction A. Therefore, for example, a part of the attachment sheet 2 that is simply covered with the device body 3, but can be separated from the device body 3 in the thickness direction A corresponds to the second part X2. Since the attachment sheet 2 includes not only the first part X1 but also the second part X2, unintended release of the attachment device 1 from a living body surface is suppressed. This will be described later in detail (see FIG. 21).

In addition, the second part X2 of the attachment sheet 2 includes an outer edge portion 13 to which the device body 3 is not fixed and that is positioned at an outer periphery of the device body 3 in plan view (see FIGS. 3 and 4) as viewed in the thickness direction A of the attachment sheet 2.

As shown in FIGS. 1 to 6, the attachment device 1 includes the plurality of release sheets 4. Specifically, the plurality of release sheets 4 include the first release sheet 41 and the second release sheet 42.

The first release sheet 41 is attached to a first attachment region including an attachment surface of the first part X1 in the attachment surface 2a of the attachment sheet 2. In other words, the first attachment region includes an attachment surface of a part of the attachment surface 2a different from the outer edge portion 13. In addition, the plurality of release sheets 4 of the present embodiment include two first release sheets 41. More specifically, the two first release sheets 41 of the present embodiment cover the whole attachment surface of the first part X1 in the attachment surface 2a of the attachment sheet 2. Further, the two first release sheets 41 of the present embodiment cover the entire region of the attachment surface of the second part X2 in the attachment surface 2a of the attachment sheet 2, excluding attachment surfaces of flap portions 12 to be described later. That is, the two first release sheets 41 of the present embodiment completely cover an attachment surface of a sheet body 11 to be described later of the attachment sheet 2.

The second release sheet 42 is attached to a second attachment region including only an attachment surface of the outer edge portion 13 of the second part X2 in the attachment surface 2a of the attachment sheet 2. In addition, the plurality of release sheets 4 of the present embodiment include two second release sheets 42. More specifically, the two second release sheets 42 of the present embodiment cover the entire regions of the attachment surfaces of the flap portions 12 to be described later in the attachment surface of the outer edge portion 13 of the second part X2.

As described above, since the attachment sheet 2 includes the second part X2, the attachment surface 2a is unlikely to peel from a living body surface (see FIG. 21). In addition, the attachment device 1 includes the second release sheet 42 in which the attachment sheet 2 is attached only to the attachment surface of the outer edge portion 13 in the second part X2. That is, in the attachment device 1, a part or the entirety (the attachment surfaces of the flap portions 12 to be described later, constituting a part of the attachment surface of the outer edge portion 13 in the present embodiment) of the attachment surface of the outer edge portion 13 of the second part X2 is covered with the second release sheet 42 different from the first release sheet 41 attached to the attachment surface of the first part X1. With such a configuration, the first release sheet 41 and the second release sheet 42 can be separately removed. That is, the first release sheet 41 and the second release sheet 42 can be peeled off from the attachment surface 2a in directions in which they are easily peeled off. Therefore, it is possible to suppress the occurrence of a problem that the outer edge portion 13 of the attachment sheet 2 deforms following the release sheet and is not separated from the release sheet.

Hereinafter, the attachment device 1 of the present embodiment will be described in further detail.

### Attachment Sheet 2

The attachment sheet 2 has flexibility. That is, the attachment sheet 2 can be relatively easily deformed in the out-of-plane direction. Therefore, the attachment sheet 2 can be deformed along the shape of a living body surface. In addition, since the attachment sheet 2 is attached to the living body surface through the attachment surface 2a, the attachment sheet 2 easily follows deformation of the living body surface. As a result, the attachment surface 2a is unlikely to peel from the living body surface, and it is possible to suppress unintended release of the attachment device 1 from the living body surface.

Means for fixing the device body 3 and the fixing surface 2b of the attachment sheet 2 is not particularly limited. The device body 3 and the fixing surface 2b of the attachment sheet 2 may be bonded to each other by, for example, adhesion, fusion, or attachment using an adhesive. The release strength between the device body 3 and the fixing surface 2b of the attachment sheet 2 is greater than at least the release strength between the release sheet 4 and the attachment surface 2a of the attachment sheet 2. The “release strength” mentioned herein can be measured by, for example, a 180° release test for the attachment sheet 2.

The attachment sheet 2 can be formed of, for example, a plurality of layers including a base layer and an adhesive layer.

The base layer can be formed of, for example, a thin resin sheet. More specifically, the base layer can be formed of, for example, a white spunlace non-woven fabric of polyester fibers, and a thickness of the base layer can range from 5 μm to 150 μm, for example, 30 μm. The material of the base layer is not limited to polyester, and may be, for example, an acrylic polymer, polyethylene, an ethylene-vinyl acetate copolymer, polyurethane, a polyamide derivative, or the like.

The adhesive layer can be formed of, for example, an adhesive such as a rubber-based adhesive, an acrylic adhesive, or a silicon-based adhesive. The adhesive layer is laminated on the base layer directly or indirectly with another layer interposed between the adhesive layer and the base layer. Therefore, the attachment surface 2a of the attachment sheet 2 of the present embodiment is constituted by the adhesive layer.

The attachment sheet 2 may further include another layer in addition to the base layer and the adhesive layer described above. The attachment sheet 2 may include, for example, a surface layer. The surface layer can be formed of, for example, a resin having a thickness of about 5 μm to 50 μm. More specifically, as the material of the surface layer, polyester, polyamide, polyamideimide, polyethylene, polypropylene, polycarbonate, polyurethane, polyvinyl chloride, fluororesin, or the like can be used. The surface layer is laminated on a side opposite to the adhesive layer with the base layer interposed between the surface layer and the adhesive layer, on the base layer directly or indirectly with another layer interposed between the surface layer and the base layer. Therefore, the upper surface of the attachment sheet 2 may be constituted by the surface layer.

More specifically, the attachment sheet 2 may be formed of a non-woven fabric tape having one surface provided with a pressure sensitive adhesive as an adhesive. Further, the attachment sheet 2 may be formed of a double-sided tape in which adhesive layers are provided on both sides of the base layer. In a case where the attachment sheet 2 is formed of a double-sided tape, the device body 3 can be fixed to the attachment sheet 2 by being adhered to one adhesive layer as the fixing surface 2b of the attachment sheet 2.

The attachment sheet 2 of the present embodiment includes the sheet body 11 extending in a substantially C-shape in plan view as viewed in the thickness direction A, and the flap portions 12 provided to extend from both opposing ends of the sheet body 11 so as to approach each other. The device body 3 of the present embodiment is fixed to the sheet body 11. Further, the device body 3 of the present embodiment is not fixed to the flap portions 12. The upper sides of the flap portions 12 are not covered with the device body 3. Therefore, the flap portions 12 are configured to be bendable and deformable in the thickness direction A with respect to the sheet body 11. A receiving portion 8 capable of receiving an insertion member for medical use that is inserted or to be inserted into a blood vessel of a living body is provided between both ends of the sheet body 11 of the present embodiment extending in a substantially C-shape. In other words, the flap portions 12 of the present embodiment are attached to the living body surface to cover at least a part of the receiving portion 8.

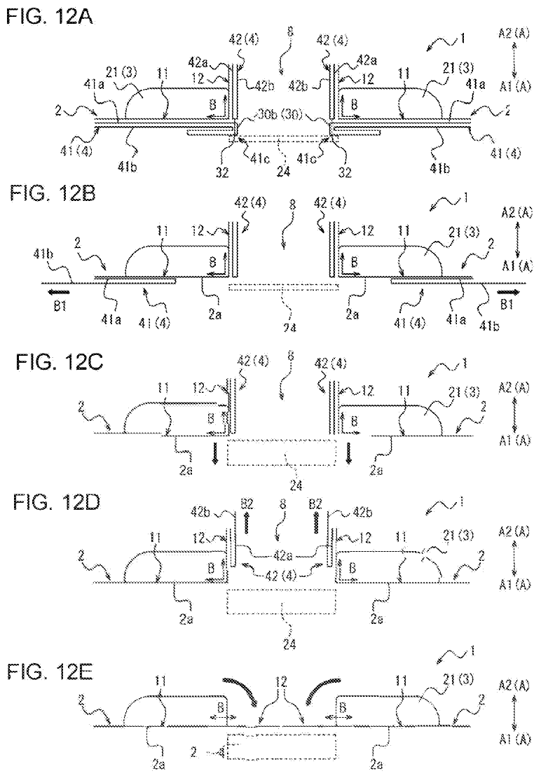

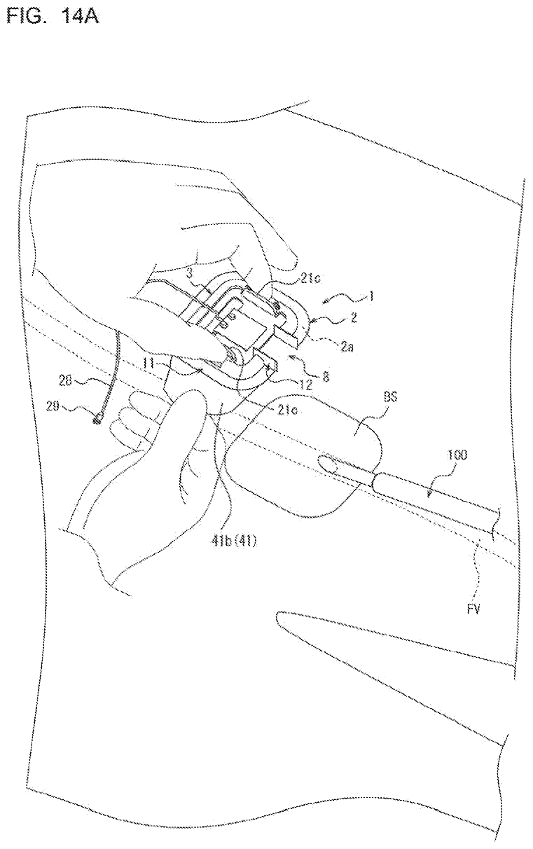

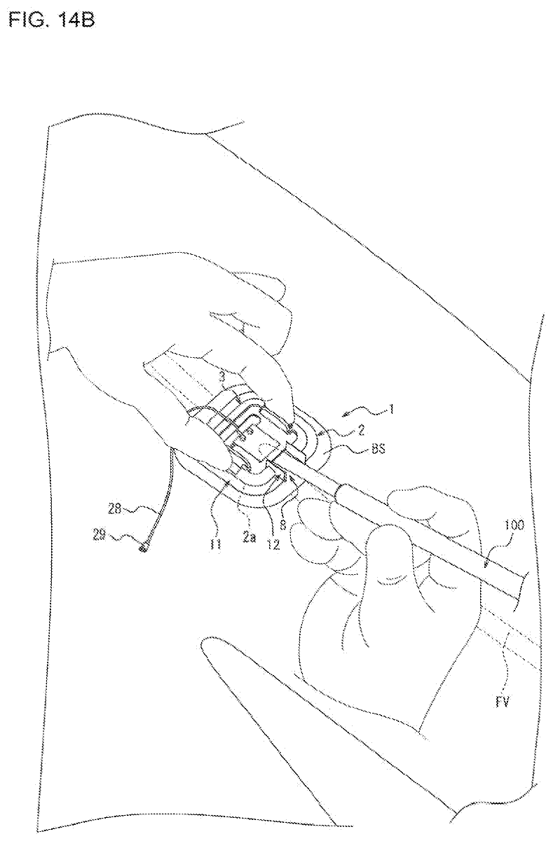

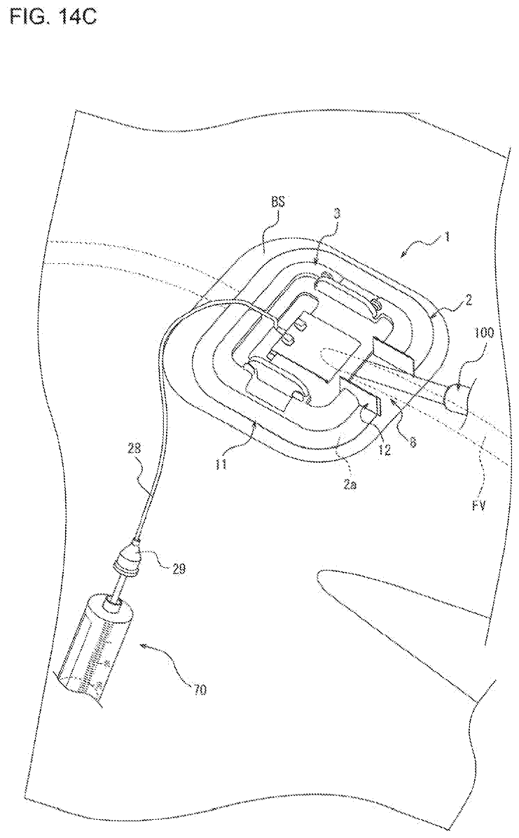

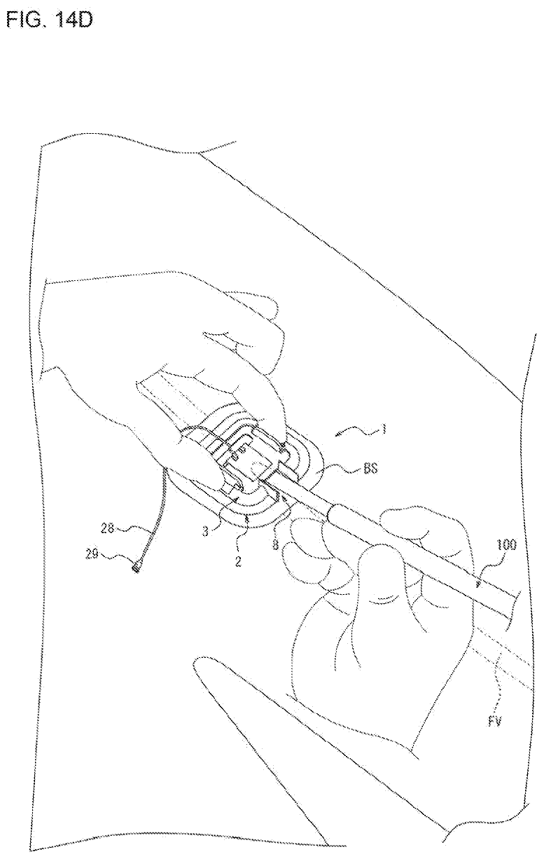

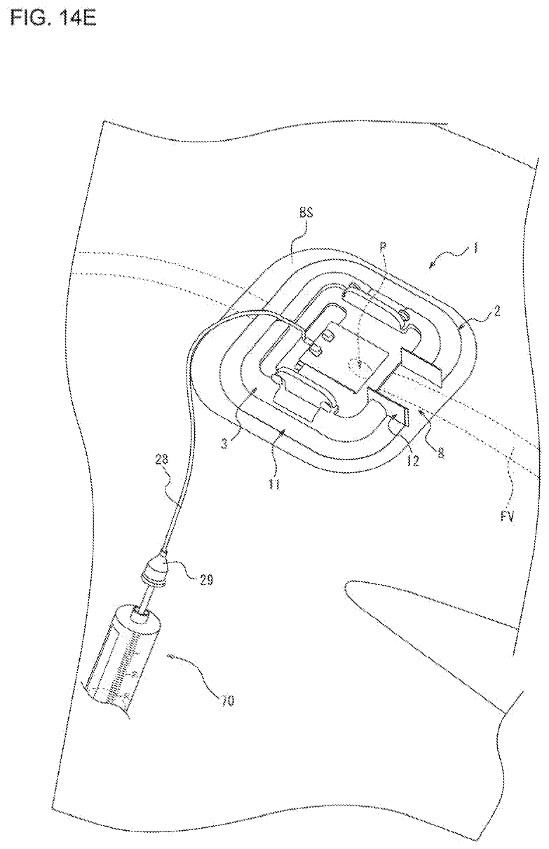

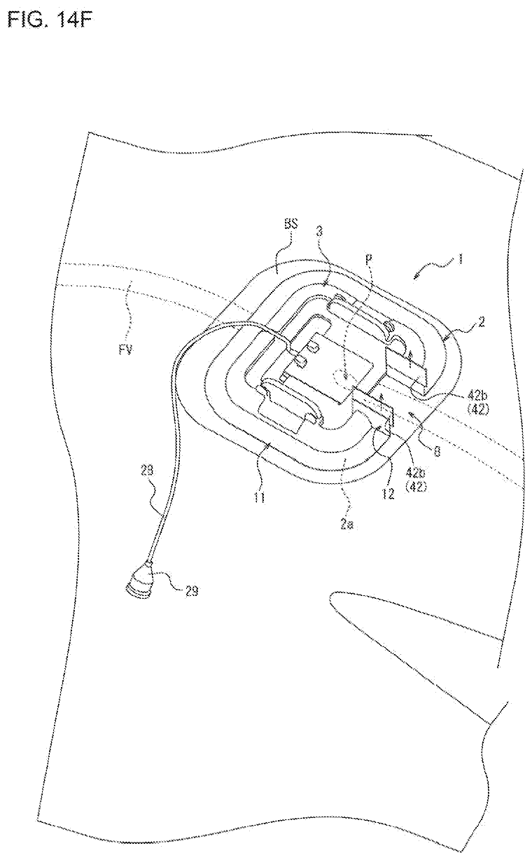

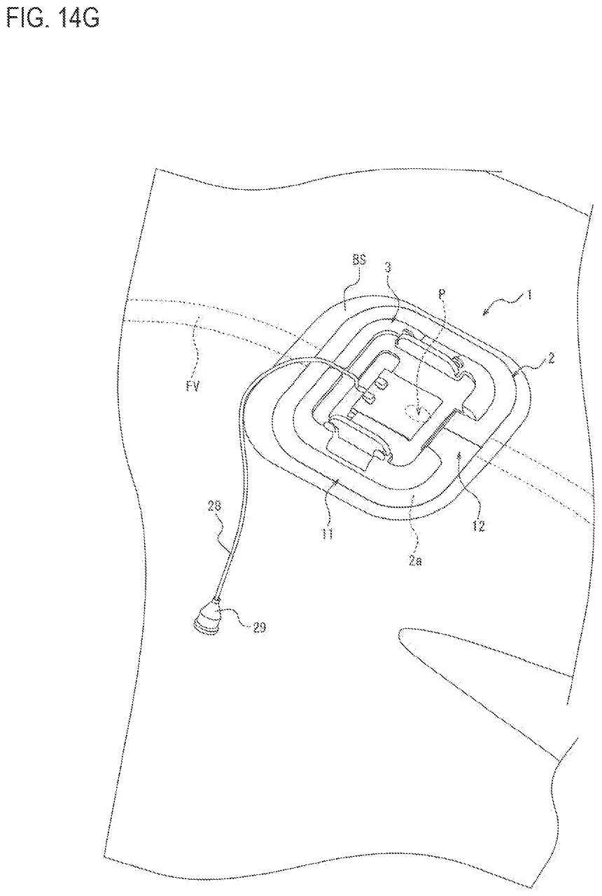

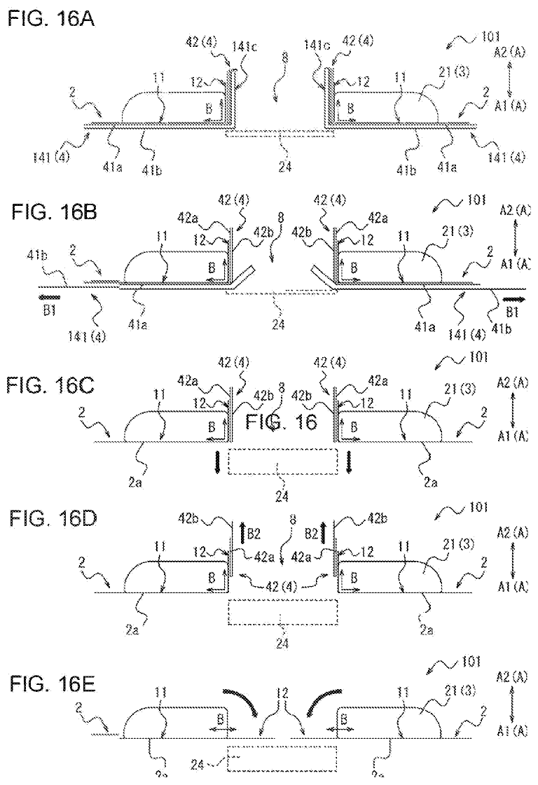

The flap portions 12 of the present embodiment are configured to cover substantially the entire region of the receiving portion 8 (see FIGS. 12E and FIG. 14G), but are not limited to this configuration. As will be described later, the flap portions 12 may be configured to cover only a part of the receiving portion 8 (see FIG. 16E).

In the present embodiment, the flap portion 12 is provided to extend from each of both opposing ends of the sheet body 11 extending in a substantially C-shape in plan view, but the present disclosure is not limited to this configuration. The flap portion 12 may be provided to extend from only one of both opposing ends of the sheet body 11 extending in a substantially C-shape in plan view. However, from the viewpoint of difficulty of peeling of the attachment sheet 2 from the living body surface, the flap portion 12 is preferably provided to extend from each of both opposing ends of the sheet body 11.

Next, the first part X1 and the second part X2 of the attachment sheet 2 will be described.

As described above, the attachment sheet 2 includes the first part X1 to which the device body 3 is fixed and the second part X2 to which the device body 3 is not fixed (see, for example, FIG. 3). The second part X2 extends continuously from the first part X1. Lower surfaces of the first part X1 and the second part X2 of the attachment sheet 2 constitute the attachment surface 2a and provided with an attachment portion formed of an adhesive or the like. Since the attachment sheet 2 of the attachment device 1 includes the above-described second part X2, it is possible to suppress unintended peeling of the attachment sheet 2 from a living body surface.

The device body 3 is fixed to the fixing surface 2b of the attachment sheet 2. Therefore, the attachment device 1 is likely to receive an external force even when attached to a living body surface. FIG. 21 shows a state in which an external force in a direction orthogonal to a living body surface BS acts on the device body 3 in a state in which the attachment surface 2a of the attachment sheet 2 is attached to the living body surface BS. As shown in FIG. 21, when an external force in a direction orthogonal to the living body surface BS acts on the device body 3, the first part X1 of the attachment sheet 2 to which the device body 3 is fixed is lifted in a direction away from the living body surface BS. Therefore, the part of the living body surface BS to which the first part X1 is attached deforms to be lifted following the first part X1. The second part X2 provided to extend from the first part X1 maintains the attachment state following the living body surface BS even in a case where the above-described deformation occurs in the living body surface BS. Therefore, a force in a direction along the attachment surface 2a and the raised part of the living body surface BS easily acts on the second part X2 provided to extend from the first part X1.

That is, it is possible to reduce the force acting in a direction orthogonal to the living body surface on the second part X2 provided to extend from the first part X1. In addition, a shearing force in a direction along the living body surface BS becomes likely to act on the second part X2 of the attachment sheet 2. The attachment sheet 2 is not easily moved and peeled off by the shearing force in a direction along the living body surface BS. By providing the second part X2 in the attachment sheet 2 as above, it is possible to realize the attachment sheet 2 that is less likely to peel from the living body surface BS than a configuration in which the second part X2 is not provided.

In particular, in a case where the attachment device 1 is a pressing device as in the present embodiment, the attachment sheet 2 preferably includes not only the first part X1 but also the second part X2. Although details will be described later, the attachment device 1 of the present embodiment is a pressing device including the pressing body 22 (see FIG. 11, etc.). That is, in the attachment device 1 of the present embodiment, the pressing body 22 can press a predetermined part of a living body surface in a state in which the attachment surface 2a is attached to the living body surface. Examples of the predetermined part on the living body surface that is pressed by the attachment device 1 can include a wound formed by inserting an insertion member for medical use such as a puncture needle, a catheter, or a sheath into a blood vessel of a living body and the vicinity of the wound. After the insertion member such as a sheath is removed from the living body, hemostasis can be achieved by pressing the wound or the vicinity of the wound on the living body surface with the pressing body 22 of the attachment device 1. The device body 3 including the pressing body 22 can maintain a state in which the living body surface is pressed by the attachment of the attachment surface 2a of the attachment sheet 2 to the living body surface. In other words, the force with which the pressing body 22 presses the living body surface acts as a force for lifting the attachment surface 2a in the upward direction A2 in the thickness direction A at the position of the first part X1 of the attachment sheet 2. That is, as shown in FIG. 21, a pulling force is likely to be generated in a direction of separating the first part X1 of the attachment sheet 2 from the living body surface BS. Therefore, in a case where the attachment device 1 is a pressing device, the attachment sheet 2 particularly preferably includes not only the first part X1 but also the second part X2.

### Device Body 3

As described above, the attachment device 1 of the present embodiment is a pressing device. Here, the device body 3 including the pressing body 22 will be described as an example, but the device body may not include the pressing body 22.

As described above, the device body 3 is fixed to the upper surface as the fixing surface 2b of the attachment sheet 2. More specifically, the device body 3 of the present embodiment covers a central region surrounded by the attachment sheet 2 extending in a substantially C-shape in plan view as viewed in the thickness direction A. The outer edge portion of the device body 3 is disposed at a position overlapping the attachment sheet 2 in the thickness direction A, and is fixed to the upper surface of the attachment sheet 2. More specifically, the outer edge portion of the device body 3 of the present embodiment is disposed at a position overlapping the sheet body 11 of the attachment sheet 2 in the thickness direction A, and is fixed to the upper surface of the sheet body 11. The device body 3 of the present embodiment does not overlap the flap portions 12 of the attachment sheet 2 in the thickness direction A. Accordingly, the device body 3 of the present embodiment is not fixed to the flap portions 12 of the attachment sheet 2. In the device body 3 of the present embodiment, the pressing body 22 to be described later can press a living body surface in the central region defined by the attachment sheet 2.

As shown in FIGS. 10 and 11, the device body 3 of the present embodiment includes a holding body 21 and the pressing body 22.

### Holding Body 21

The holding body 21 is fixed to the fixing surface 2b of the attachment sheet 2. In addition, the holding body 21 holds the pressing body 22.

As shown in FIG. 3, the holding body 21 of the present embodiment covers a central region surrounded by the attachment sheet 2 extending in a substantially C-shape in plan view. The outer edge portion of the holding body 21 is disposed at a position overlapping the sheet body 11 of the attachment sheet 2 in the thickness direction A, and is fixed to the upper surface of the sheet body 11.

More specifically, the holding body 21 of the present embodiment includes a frame portion 21a, a cover portion 21b, and a gripping portion 21c. The frame portion 21a extends in a substantially C-shape in plan view. The frame portion 21a is fixed to the upper surface of the sheet body 11 of the attachment sheet 2. The cover portion 21b is connected to the inner side of the frame portion 21a in plan view. The cover portion 21b covers a central region surrounded by the attachment sheet 2 having a substantially C-shape in plan view. The cover portion 21b can be formed of a plate-like portion that is flat in the thickness direction A. The gripping portion 21c protrudes from the cover portion 21b in the upward direction A2. The gripping portion 21c of the present embodiment is formed of two gripping plate portions protruding from two opposing portions of the cover portion 21b in plan view in the upward direction A2. The two gripping plate portions as the gripping portion 21c are disposed to face each other. An operator such as a medical worker who uses the attachment device 1 can hold the attachment device 1 by gripping the two gripping plate portions disposed to face each other so that the two gripping plate portions are sandwiched in between the fingers of the operator.

Examples of the material of the holding body 21 include a resin material. Examples of the resin material include thermoplastic resins used in injection molding such as ABS resin, AS resin, polyethylene, polypropylene, polystyrene, polyvinyl chloride, polyvinylidene chloride resin, polyphenylene oxide, thermoplastic polyurethane, polymethylene methacrylate, polyoxyethylene, fluororesin, polycarbonate, polyamide, acetal resin, acrylic resin, and polyethylene terephthalate, and thermosetting resins such as phenol resin, epoxy resin, silicone resin, and unsaturated polyester.

### Pressing Body 22

The pressing body 22 is held by the holding body 21. The pressing body 22 can be protruded from the attachment surface 2a of the attachment sheet 2 in the downward direction A1 as one side in the thickness direction A. In the attachment device 1, the pressing body 22 is protruded in the downward direction A1 from the attachment surface 2a of the attachment sheet 2 in a state in which the attachment surface 2a of the attachment sheet 2 is attached to a living body surface. As a result, the living body surface is pressed by the pressing body 22.

The pressing body 22 of the present embodiment includes the expansion portion 24 that is expandable in the downward direction A1 in the thickness direction A by supply of a fluid such as air. In a case where the expansion portion 24 of the present embodiment is changed from the contracted state shown in FIG. 10 to the expanded state shown in FIG. 11, it protrudes in the downward direction A1 from the attachment surface 2a of the attachment sheet 2 in the thickness direction A, and has a posture so as to press the living body surface.

Specifically, the attachment device 1 of the present embodiment is attached on the living body surface in a state in which the expansion portion 24 of the pressing body 22 is in a contracted state (see FIG. 10) before expansion. The expansion portion 24 is expanded by an operation of an operator such as a medical worker and enters an expanded state (see FIG. 11) in a state in which the attachment device 1 is attached on the living body surface. Therefore, the attachment device 1 of the present embodiment can press a predetermined part on the living body surface with the expansion portion 24. As described above, the predetermined part on the living body surface can be, for example, a wound or the like formed by inserting an insertion member for medical use into a blood vessel of a living body. After the insertion member such as a sheath is removed from the living body, hemostasis can be achieved by pressing the wound or the vicinity of the wound on the living body surface with the expansion portion 24 of the pressing body 22 of the attachment device 1. The series of living body pressing methods will be described later in detail (see FIGS. 14A to 14G).

As shown in FIG. 10, the expansion portion 24 of the pressing body 22 of the present embodiment in a contracted state is substantially flush with the attachment surface 2a of the attachment sheet 2, and does not protrude at all or hardly in the downward direction A1 from the attachment surface 2a.

The expansion portion 24 of the present embodiment defines an internal space 24a. The internal space 24a of the expansion portion 24 communicates with a tube 28 extending to the outside of the holding body 21. A fluid such as air is supplied to the internal space 24a of the expansion portion 24 from a fluid feeder such as a syringe connected to an inflation port as a connection portion 29 provided at an end portion of the tube 28 through the tube 28. Therefore, the expansion portion 24 can be changed from the contracted state (see FIG. 10) to the expanded state (see FIG. 11). That is, the expansion portion 24 of the pressing body 22 can be protruded in the downward direction A1 from the attachment surface 2a of the attachment sheet 2. The fluid supplied to the internal space 24a of the expansion portion 24 is not limited to the gas and may be a liquid. The expansion portion 24 can be changed from the expanded state (see FIG. 11) to the contracted state (see FIG. 10) by suctioning or removing the fluid from the internal space 24a of the expansion portion 24.

More specifically, as shown in FIG. 11, the expansion portion 24 of the present embodiment includes a first balloon portion 25a that is brought into contact with and directly presses a living body surface, and a second balloon portion 25b that presses the first balloon portion 25a in the downward direction A1 to increase a pressing force of the first balloon portion 25a in the downward direction A1.

The first balloon portion 25a defines a first internal space 25a1. The second balloon portion 25b defines a second internal space 25b1. The first internal space 25a1 and the second internal space 25b1 communicate with each other. In the contracted state (see FIG. 10) of the expansion portion 24, the first balloon portion 25a and the second balloon portion 25b in the contracted state overlap each other in the thickness direction A. The first balloon portion 25a in the contracted state is positioned on the lower side, and the second balloon portion 25b in the contracted state is overlapped on the upper side. The second balloon portion 25b is covered with the holding body 21 in the upward direction A2. The first balloon portion 25a and the second balloon portion 25b are continuous with each other only on one end side (left side in FIGS. 10 and 11) in a direction orthogonal to the thickness direction A.

In addition, the pressing body 22 of the present embodiment includes an extension portion 26 that is provided to extend from the expansion portion 24 and wound from the lower side to the upper side of the holding body 21 through a through hole 21b1 of the holding body 21. The extension portion 26 is locked to the upper surface of the holding body 21.

As a material constituting the pressing body 22 of the present embodiment, for example, soft polyvinyl chloride, polyurethane, polyethylene, polypropylene, polyester, an ethylene-vinyl acetate copolymer (EVA), silicone, or a flexible material obtained by mixing any of these materials can be used.

The configuration of the pressing body 22 is not limited to the configuration of the present embodiment. The expansion portion 24 of the pressing body 22 may include, for example, only one balloon portion. The configuration of the pressing body 22 is not particularly limited as long as it is held by the holding body 21 and can press a living body surface.

### First Release Sheet 41 and Second Release Sheet 42

The first release sheet 41 of the present embodiment includes a release layer 41a and a folding layer 41b. In addition, the second release sheet 42 of the present embodiment includes a release layer 42a and a folding layer 42b. Hereinafter, for explanatory convenience, the release layer 41a of the first release sheet 41 is referred to as “first release layer 41a,” and the release layer 42a of the second release sheet 42 is referred to as “second release layer 42a.” In addition, the folding layer 41b of the first release sheet 41 is referred to as “first folding layer 41b,” and the folding layer 42b of the second release sheet 42 is referred to as “second folding layer 42b.”

First, the configuration of each of the first release sheet 41 and the second release sheet 42 of the present embodiment will be described. As shown in FIG. 7, the first release sheet 41 of the present embodiment is folded once from an original single layer form (the left view in FIG. 7) to be in an intermediate two-layer form (the middle view in FIG. 7). Furthermore, a part of the first release sheet 41 of the present embodiment is further folded from the intermediate two-layer form to be in a complete form (the right view in FIG. 7) in which a part is four layered and the other part is two layered. In this manner, the first release sheet 41 that includes the first release layer 41a consisting of one layer and the first folding layer 41b of which a part consists of three layers and the other part consists of one layer is formed.

As shown in FIG. 7, the first release sheet 41 of the present embodiment has two linear folds 30 at the outer edge of the first release layer 41a. In addition, the first release sheet 41 of the present embodiment includes a fold 31 between the first folding layers 41b, separately from the folds 30. Hereinafter, for explanatory convenience, of the two folds 30, a fold far from the fold 31 between the first folding layers 41b is referred to as “first fold 30a.” In addition, of the two folds 30, a fold close to the fold 31 between the first folding layers 41b is referred to as “second fold 30b.”

The configurations of the first release layer 41a and the first folding layer 41b of the first release sheet 41 are not limited to the configurations shown in FIG. 7. In the present embodiment, the complete form in FIG. 7 (the right view in FIG. 7) is used as the first release sheet 41, but for example, the intermediate form in FIG. 7 (the middle view in FIG. 7) may be used as the first release sheet. In other words, the first folding layer 41b may consist only of one layer or consist of a plurality of layers. In the present embodiment, the receiving portion 8 (see FIG. 1) capable of receiving an insertion member such as a sheath is secured by forming a part from the intermediate form in FIG. 7 into the complete folded form.

Further, in the present embodiment, as shown, for example, in FIG. 2, two first release sheets 41 are attached to the attachment surface 2a of one attachment sheet 2, however, for example, three or more first release sheets may be attached. That is, the shape of each first release sheet and the number of the first release sheets attached are not particularly limited.

The first folding layer 41b of the first release sheet 41 constitutes a sheet gripping portion that is gripped when the first release sheet 41 is released from the attachment sheet 2. As in the present embodiment, the first folding layer 41b as a sheet gripping portion may be constituted only by a part overlapping the first release layer 41a in the layer thickness direction. The first release sheet 41 may further include a tab provided to extend from the outer edge of the first folding layer 41b to a position that does not overlap the first release layer 41a in the layer thickness direction.

As shown in FIG. 8, the second release sheet 42 of the present embodiment is folded once from an original single layer form (the left view in FIG. 8) to be in a complete two-layer form (the right view in FIG. 8). In this manner, the second release sheet 42 that includes the second release layer 42a consisting of one layer and the second folding layer 42b consisting of one layer is formed.

As shown in FIG. 8, the second release sheet 42 of the present embodiment has one linear fold 32 at the outer edge of the second release layer 42a. The second folding layer 42b of the present embodiment is folded on the second release layer 42a via the fold 32.

The configurations of the second release layer 42a and the second folding layer 42b of the second release sheet 42 are not limited to the configurations shown in FIG. 8. For example, the second release sheet 42 may include the second folding layer 42b consisting only of one layer as in the present embodiment, or may include the second folding layer 42b consisting of a plurality of layers.

Further, in the present embodiment, as shown, for example, in FIG. 2, two second release sheets 42 are attached to the attachment surface 2a of one attachment sheet 2, however, for example, three or more second release sheets may be attached. That is, the shape of each second release sheet and the number of the second release sheets attached are not particularly limited.

The second folding layer 42b of the second release sheet 42 constitutes a sheet gripping portion that is gripped when the second release sheet 42 is released from the attachment sheet 2. As in the present embodiment, the second folding layer 42b as a sheet gripping portion may be constituted only by a part overlapping the second release layer 42a in the layer thickness direction. The second release sheet 42 may further include a tab provided to extend from the outer edge of the second folding layer 42b to a position that does not overlap the second release layer 42a in the layer thickness direction.

Next, positions of the first release sheet 41 and the second release sheet 42 of the present embodiment attached to the attachment surface 2a of the attachment sheet 2 will be described.

In the first release sheet 41 of the present embodiment, the first release layer 41a is attached to the attachment surface 2a of the attachment sheet 2. A region where the first release layer 41a is attached in the attachment surface 2a is referred to as “first attachment region.” The first attachment region covered by the first release layers 41a of the two first release sheets 41 of the present embodiment is the whole region of the attachment surface 2a of the attachment sheet 2, excluding the attachment surfaces of the flap portions 12. More specifically, the first attachment region covered by the first release layers 41a of the two first release sheets 41 of the present embodiment is the entire region of the attachment surface of the sheet body 11 in the attachment surface 2a of the attachment sheet 2. In the first release sheet 41 of the present embodiment, the first folding layer 41b is folded and laminated on the first release layer 41a on a side of the attachment surface 2a opposite to the first attachment region side.

As shown in FIG. 4, the first fold 30a, that is one of the two folds 30 between the first release layer 41a and the first folding layer 41b of the first release sheet 41, passes through the first part X1 of the attachment sheet 2 in plan view. In other words, at least a part of the first fold 30a overlaps the first part X1 of the attachment sheet 2 in plan view. The first release layer 41a of the first release sheet 41 is released from the attachment surface 2a of the attachment sheet 2 by moving the first folding layer 41b. Therefore, the first release layer 41a is released from the position of the fold 30 on the attachment surface 2a of the attachment sheet 2. With a configuration in which the first fold 30a passes through the first part X1 of the attachment sheet 2 in plan view, the position of the first part X1 in the attachment sheet 2 can be provided as a position where the release of the first release layer 41a of the first release sheet 41 is started. By starting the release of the first release layer 41a of the first release sheet 41 from the first part X1 of the attachment sheet 2, the first release sheet 41 is more easily released from the attachment sheet 2 than in a case where the release is started from the second part X2 of the attachment sheet 2. That is, it is possible to suppress winding and bending of the second part X2 of the attachment sheet 2 due to the release operation of the first release sheet 41.

The first fold 30a of the present embodiment passes through the first part X1 of the attachment sheet 2 in plan view as shown in FIG. 4, but may be a fold that does not pass through the first part X1 and the second part X2 of the attachment sheet 2 in plan view. Even in a case where the first fold is such a fold, the same effects as described above can be obtained in a case where the second part X2 is not interposed between the fold and the first part X1 of the attachment sheet 2 in plan view. The same effects as described above can also be obtained in a case where, although the second part X2 is interposed between the fold and the first part X1 of the attachment sheet 2 in plan view, the length thereof is sufficiently short (for example, 2 mm or less).

As shown in FIG. 4, the second fold 30b, that is the other of the two folds 30 between the first release layer 41a and the first folding layer 41b of the first release sheet 41, does not pass through the first part X1 of the attachment sheet 2 in plan view. However, the second fold 30b does not pass through the first part X1 and the second part X2 of the attachment sheet 2 in plan view. In addition, the second part X2 is not interposed between the second fold 30b and the first part X1 of the attachment sheet 2 in plan view. Therefore, the same effects as those of the first fold 30a can also be obtained at the position of the second fold 30b.

Therefore, by moving the first folding layer 41b of the present embodiment away from the two linear folds 30 in a direction (the horizontal direction in FIG. 4) orthogonal to the two folds 30 in plan view (see FIG. 4, etc.), the first release layer 41a can be released from the attachment surface 2a without winding and bending of the second part X2 of the attachment sheet 2.

In the second release sheet 42 of the present embodiment, the second release layer 42a is attached to the attachment surface 2a of the attachment sheet 2. A region where the second release layer 42a is attached in the attachment surface 2a is referred to as “second attachment region.” The second attachment region covered by the second release layers 42a of the two second release sheets 42 of the present embodiment is the entire region of the attachment surfaces of the flap portions 12 in the attachment surface 2a of the attachment sheet 2. In the second release sheet 42 of the present embodiment, the second folding layer 42b is folded and laminated on the second release layer 42a on a side of the attachment surface 2a opposite to the second attachment region side.

As described above, the second release layer 42a of the second release sheet 42 of the present embodiment is attached only to the attachment surface of the flap portion 12 in the attachment surface 2a of the attachment sheet 2. Further, the fold 32 is disposed on the sheet body 11 side of the flap portion 12. Therefore, by moving the second folding layer 42b of the present embodiment away from the fold 32 (in the upward direction in FIG. 6) in a direction orthogonal to the fold 32 (in the vertical direction in FIG. 6) in the in-plane direction of the flap portion 12, the second release layer 42a can be released from the attachment surface 2a without winding and bending of the flap portion 12 that is a part of the second part X2 of the attachment sheet 2.

As above, since the attachment device 1 of the present embodiment includes the first release sheet 41 and the second release sheet 42 described above, it is possible to suppress the occurrence of winding and bending of the second part X2 of the attachment sheet 2 during the operation of releasing the first release sheet 41 and the second release sheet 42.

Furthermore, in the attachment device 1 of the present embodiment, as described later, an insertion member such as a sheath can be received from the receiving portion 8 by lifting the flap portions 12 without release of the second release sheets 42 from the flap portions 12 (see FIG. 14B). The second release sheet 42 is released from the attachment surface of the flap portion 12 after the insertion member such as a sheath is removed from a living body (see FIG. 14F).

Here, in the present embodiment, the second attachment region of the attachment surface 2a where the second release layer 42a of the second release sheet 42 is attached is adjacent to the first attachment region of the attachment surface 2a where the first release layer 41a of the first release sheet 41 is attached. More specifically, in the present embodiment, the attachment surface of the flap portion 12 where the second release layer 42a of the second release sheet 42 is attached is adjacent to the attachment surface of the sheet body 11 where the first release layer 41a of the first release sheet 41 is attached.

In addition, the first release sheet 41 of the present embodiment includes an overlapping portion 41c that covers the second release sheet 42. By providing the overlapping portion 41c, it is possible to suppress the exposure of the attachment surface 2a between the first release sheet 41 and the second release sheet 42.

In the present embodiment, the first release sheet 41 includes the overlapping portion 41c, but the second release sheet 42 may include an overlapping portion that covers the first release sheet 41. However, as in the present embodiment, the first release sheet 41 preferably includes the overlapping portion 41c. As shown in FIG. 6, in the present embodiment, the boundary between the first attachment region (the attachment surface of the sheet body 11 in the present embodiment) and the second attachment region (the attachment surface of the flap portion 12 in the present embodiment) is defined by a ridgeline formed by bending of the outer edge portion 13 of the attachment sheet 2 toward the fixing surface 2b side (upper side). In other words, in the present embodiment, the flap portion 12 is bent upward with respect to the sheet body 11. Therefore, in a case where the second release sheet 42 includes an overlapping portion, the overlapping portion protrudes in the downward direction A1 from the first release sheet 41. Therefore, there is a concern that the overlapping portion of the second release sheet 42 may come into contact with a living body surface and cause discomfort to a patient. In the present embodiment, since the first release sheet 41 includes the overlapping portion 41c, the above-described discomfort to a patient of the overlapping portion of the second release sheet 42 coming into contact with the living body surface can be avoided.

More specifically, as shown in FIG. 6, the overlapping portion 41c of the first release sheet 41 of the present embodiment protrudes in a direction away from the second release sheet 42 (the direction toward the inside of the receiving portion 8 in the horizontal direction in FIG. 6) along the first attachment region (the attachment surface of the sheet body 11 in the present embodiment), not along the second attachment region (the attachment surface of the flap portion 12 in the present embodiment). However, as described later, the overlapping portion 41c of the first release sheet 41 may extend along the second attachment region (the attachment surface of the flap portion 12 in the present embodiment) and cover the second release sheet 42 (see FIG. 16).

As the release sheet 4, a plastic film such as polyethylene, polyvinyl chloride, polyester, or polypropylene, paper, a metal film, cloth, or the like can be used. In addition, the release sheet 4 may be a single layer film consisting of one film layer, or a laminated film in which a plurality of types of films are laminated. Furthermore, the surface of the release sheet 4 attached to the attachment surface 2a of the attachment sheet 2 may be subjected to a release treatment such as a silicone treatment or embossing. The thickness of the release sheet 4 can be, for example, 15 μm to 200 μm, and preferably 40 μm to 100 μm.

In the present embodiment, the tensile modulus of elasticity of the release sheet 4 is less than the tensile modulus of elasticity of the attachment sheet 2. That is, both the attachment sheet 2 and the release sheet 4 is flexible, but in the present embodiment, the attachment sheet 2 has higher flexibility and is more easily bent than the release sheet 4.

### Living Body Pressing Method That is Performed Using Attachment Device 1

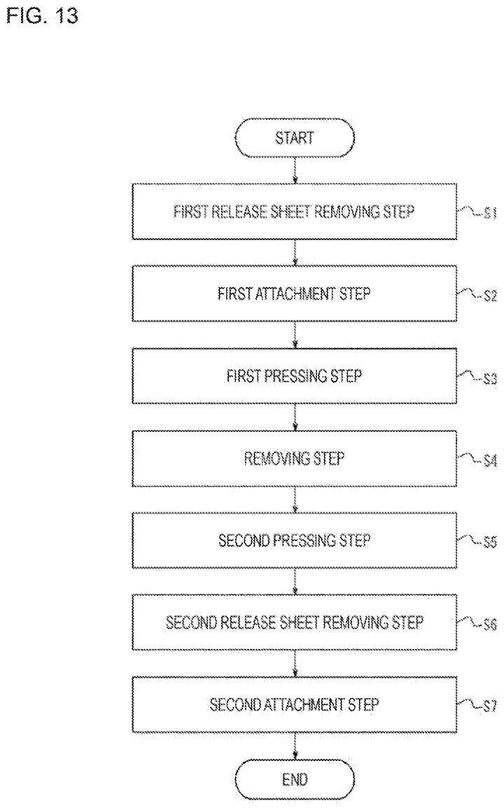

Next, a living body pressing method that is performed on a living body surface using the attachment device 1 will be described. FIGS. 12A to 12E are explanatory views explaining a procedure for removing the first release sheet 41 and the second release sheet 42 in the living body pressing method that is performed on a living body surface using the attachment device 1. FIG. 13 is a flowchart showing an example of the living body pressing method that is performed on a living body surface using the attachment device 1. The living body pressing method shown in FIG. 13 includes a method of attaching the attachment device 1. More specifically, the living body pressing method shown in FIG. 13 includes a first release sheet removing step S1, a first attachment step S2, a first pressing step S3, a removing step S4, a second pressing step S5, a second release sheet removing step S6, and a second attachment step S7. FIG. 14A is a view showing an outline of the first release sheet removing step S1. FIG. 14B is a view showing an outline of the first attachment step S2. FIG. 14C is a view showing an outline of the first pressing step S3. FIG. 14D is a view showing an outline of the removing step S4. FIG. 14E is a view showing an outline of the second pressing step S5. FIG. 14E is a view showing an outline of the second release sheet removing step S6. FIG. 14G is a view showing an outline of the second attachment step S7.

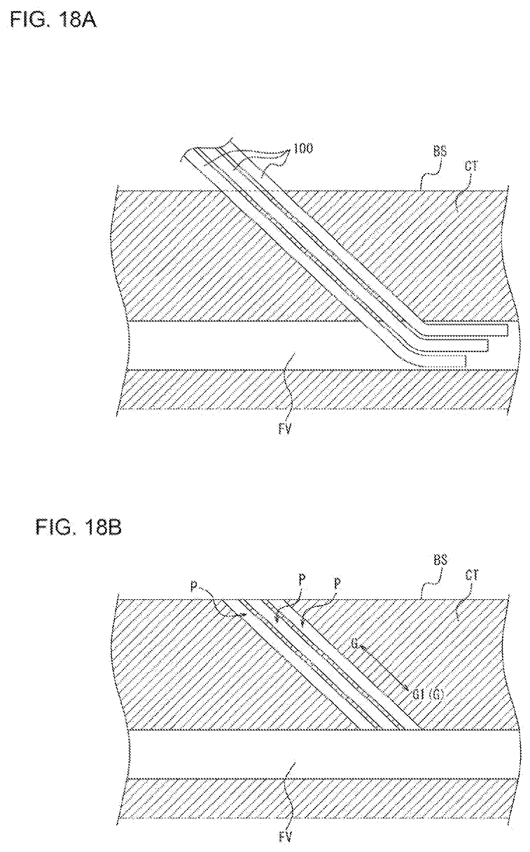

The living body pressing method shown in FIGS. 12A to 12E, 13, and 14A to 14G is a living body pressing method of pressing a living body surface BS to narrow or occlude a perforation leading from the living body surface to a vein without occluding the vein. The perforation is formed by removing a sheath as an insertion member 100 for medical use in a state inserted from the living body surface BS into a vein such as a femoral vein through a connective tissue. By the living body pressing method shown here, hemostasis after removal of the sheath as the insertion member 100 can be achieved. First, a perforation that is formed after removal of an insertion member 100 will be described with reference to FIGS. 18A and 18B. FIG. 18A shows a state in which sheaths as insertion members 100 are inserted from the living body surface BS into a femoral vein FV through a connective tissue CT. Although FIG. 18A shows three sheaths as the insertion members 100, the number of sheaths may be two or less or four or more. FIG. 18B shows a state after removal of the sheaths as the insertion members 100 from the state shown in FIG. 18A. As shown in FIG. 18B, perforations P are formed between the living body surface BS and the femoral vein FV by removing the sheaths as the insertion members 100. In the living body pressing method shown in FIGS. 12A to 12E, 13, and 14A to 14G, the perforation P can be narrowed or occluded without occluding the femoral vein FV. Therefore, even in hemostasis of bleeding from a vein at a deep position from the living body surface, it is not necessary to narrow or occlude the vein itself, and hemostasis can be achieved more efficiently. Hereinafter, details of steps S1 to S7 will be described with reference to FIGS. 12A to 12E and 14A to 14G.

FIG. 14A shows a state in which a sheath as an insertion member 100 is inserted from a living body surface BS into a femoral vein FV (see FIGS. 18A and 18B). First, in this state, the attachment device 1 is attached on the living body surface BS.

As shown in FIGS. 12A, 12B, and 14A, first, an operator such as a medical worker releases the first release sheet 41 from the attachment sheet 2. As shown in FIG. 14A, in a state in which the operator holds the device body 3 by one hand, the operator releases the first release sheet 41 by using the other hand. For example, in a state in which the operator grips the gripping portion 21c by one end, the operator grips the first folding layer 41b of the first release sheet 41 by the other hand and moves the first folding layer 41b along the first release layer 41a. Therefore, the first release layer 41a of the first release sheet 41 can be removed from the attachment surface of the sheet body 11 in the attachment surface 2a of the attachment sheet 2.

Next, as shown in FIG. 14B, the attachment surface 2a (the attachment surface of the sheet body 11 as the first attachment region in the present embodiment) of the attachment sheet 2 exposed by removing the first release sheet 41 is attached to the living body surface BS. In this case, it is preferable that the flap portions 12 of the attachment sheet 2 to which the second release sheets 42 are attached be bent with respect to the sheet body 11 and be maintained in a state of being lifted in the upward direction A2. In this way, the insertion member 100 can be easily received in the receiving portion 8.

Next, as shown in FIGS. 12C and 14C, a syringe 70 as a fluid feeder is connected to the connection portion 29 of the tube 28. Air is supplied to the internal space 24a (see FIG. 11) of the expansion portion 24 (see FIG. 11) of the attachment device 1 through the tube 28 to expand the expansion portion 24. In this way, it is possible to previously press the vicinity of the wound on the living body surface BS before removing the sheath as the insertion member 100 for medical use from the living body surface BS. In other words, pressing the living body surface BS is started in a state in which the sheath as the insertion member 100 is inserted from the living body surface BS into the femoral vein FV as a vein through the connective tissue CT (see FIGS. 18A and 18B). In this manner, pressing is performed before the insertion member 100 is removed from the living body surface BS. Therefore, the living body surface BS can be pressed immediately after removal of the sheath as the insertion member 100. Accordingly, the perforation P (see FIG. 18B) extending from the living body surface BS to the femoral vein FV (see FIGS. 18A and 18B) can be narrowed or occluded immediately after removal of the sheath.

Next, as shown in FIG. 14D, the sheath as the insertion member 100 is removed from the living body surface BS. Due to the removal of the sheath, the perforation P shown in FIG. 18B is formed. In this state, in a case where the living body surface BS is not pressed at all, bleeding occurs from the femoral vein FV to the outside of the living body through the perforation P and the wound on the living body surface BS. However, in the living body pressing method shown here, as shown in FIG. 14C, the living body surface BS is previously pressed before the sheath as the insertion member 100 is removed from the living body surface BS. Therefore, the living body surface BS can be pressed immediately after removal of the sheath so that the perforation P (see FIG. 18B) is narrowed or occluded, and the amount of bleeding immediately after removal of the sheath can be suppressed.

Next, as shown in FIG. 14E, the syringe 70 as a fluid feeder is connected again to the connection portion 29 of the tube 28. Through the tube 28, air is supplied again to the internal space 24a (see FIG. 11) of the expansion portion 24 (see FIG. 11) of the attachment device 1 to increase the pressure, or is removed to reduce the pressure. In other words, the pressing force on the living body surface BS is adjusted after removal of the sheath as the insertion member 100. Accordingly, the perforation P (see FIG. 18B) is further narrowed or occluded without occluding the femoral vein FV (see FIGS. 18A and 18B) by adjusting the pressing force on the living body surface BS, and thus it is possible to greatly reduce the amount of bleeding or stop the bleeding.

More specifically, in a case where bleeding is confirmed after removal of the sheath, the pressing force is slowly increased to increase the pressure until hemostasis is achieved. In contrast, in a case where hemostasis is confirmed after removal of the sheath, the pressing force is slowly reduced to reduce the pressure until bleeding is confirmed. Then, after bleeding is confirmed, the pressing force is slowly increased to increase the pressure until hemostasis is achieved. In this way, it is possible to suppress the occlusion of the femoral vein FV (see FIGS. 18A and 18B) due to excessive pressurization.

Next, as shown in FIGS. 12D and 14F, the second release sheet 42 is released from the attachment sheet 2. As shown in FIGS. 12D and 14F, the second release sheet 42 is released in a state in which the first attachment region (the attachment surface of the sheet body 11 in the present embodiment) of the attachment surface 2a of the attachment sheet 2 is attached to the living body surface BS. For example, in a state in which the operator presses the device body 3 on the living body surface BS by one end, the operator grips the second folding layer 42b of the second release sheet 42 with the other hand and moves the second folding layer 42b along the second release layer 42a. More specifically, the operator moves the second folding layer 42b of the second release sheet 42 in the upward direction A2 orthogonal to the living body surface BS. Therefore, the second release layer 42a of the second release sheet 42 can be removed from the attachment surface of the flap portion 12 in the attachment surface 2a of the attachment sheet 2. In other words, by moving the second release sheet 42 in a direction orthogonal to the first attachment region (the attachment surface of the sheet body 11 in the present embodiment) in a state attached to the living body surface BS, the second release sheet 42 is wound and peeled off from the second attachment region (the attachment surface of the flap portion 12 in the present embodiment). In this manner, the second release sheet 42 is released from the second attachment region (the attachment surface of the flap portion 12 in the present embodiment).

In addition, as shown in FIGS. 12B and 12D, a first folding direction B1 of the first release sheet 41 from the first release layer 41a to the first folding layer 41b is different from a second folding direction B2 of the second release sheet 42 from the second release layer 42a to the second folding layer 42b in an in-plane direction B of the attachment surface 2a. As shown in FIG. 12B, the first release layer 41a of the first release sheet 41 is peeled off from the attachment surface 2a by moving the first folding layer 41b in the first folding direction B1. In addition, as shown in FIG. 12D, the second release layer 42a of the second release sheet 42 is peeled off from the attachment surface 2a by moving the second folding layer 42b in the second folding direction B2. That is, since the first folding direction B1 is different from the second folding direction B2, the peeling direction of the first release layer 41a and the peeling direction of the second release layer 42a can be made different. Therefore, in the present embodiment, by making the peeling direction (the upward direction in FIGS. 12A to 12E) of the second release layer 42a of the second release sheet 42 different from the peeling direction (the horizontal direction in FIGS. 12A to 12E) of the first release layer 41a of the first release sheet 41 in the in-plane direction B of the attachment surface 2a, it is possible to suppress the occurrence of winding and bending of the flap portion 12 when the second release layer 42a is released from the flap portion 12.

More specifically, in the present embodiment, as shown in FIGS. 12B and 12D, the first folding direction B1 and the second folding direction B2 are in mutually opposite directions in the in-plane direction B of the attachment surface 2a of the attachment sheet 2. In this way, the attachment sheet 2 is unlikely to be twisted when the first release sheet 41 and the second release sheet 42 are released from the attachment surface 2a.

Finally, as shown in FIG. 14G, the second attachment region (the attachment surface of the flap portion 12 in the present embodiment) of the attachment surface 2a of the attachment sheet 2 exposed by removing the second release sheet 42 is attached to the living body surface BS. Since the insertion member 100 has already been removed from the living body (see FIG. 14D), the attachment surface of the flap portion 12 is attached to the living body surface BS without interfering with the insertion member 100 at the position of the receiving portion 8.

Hemostasis can be completed by maintaining the pressing state for several hours (for example, 2 hours to 6 hours). After completion of hemostasis, the attachment surface 2a of the attachment sheet 2 is peeled off from the living body surface BS to detach the attachment device 1 from the living body surface BS.

As described above, in the living body pressing method shown here, the perforation P (see FIG. 18B) is narrowed or occluded without occluding the femoral vein FV (see FIGS. 18A and 18B). In hemostasis of the vein, hemostasis can be achieved by narrowing or occluding the perforation P (see FIG. 18B). Regarding this, for example, in hemostasis of the femoral artery, even in a case where only the perforation is occluded, hemostasis cannot be achieved since blood leaks and spreads into the connective tissue CT (see FIGS. 18A and 18B). In hemostasis of the femoral artery, it is necessary to adopt a large-scale measure such as a method of strongly pressing the artery so as to narrow or occlude the artery itself and a method of blocking the hole of an artery wall.

Therefore, in the above-described living body pressing method, the living body surface BS is preferably pressed to a position where the pressing depth from the living body surface BS is, for example, 5 mm to 20 mm. By setting the pressing depth within the above range, the pressing state in which the perforation P (see FIG. 18B) is narrowed or occluded can be easily realized without occluding the vein. The pressing depth, for example, can be preferably 5 mm to 15 mm, and more preferably 8 mm to 12 mm.

Furthermore, in the above-described living body pressing method, the living body surface BS, for example, can be preferably pressed at 10 g/cm² to 600 g/cm². The pressing pressure is a pressure after removal of the sheath as the insertion member 100, and does not mean the above-described pressing force before removal of the sheath. By setting the pressing pressure within the above range, the pressing state in which the perforation P (see FIG. 18B) is narrowed or occluded is easily realized without occluding the vein. The pressing pressure can be, for example, preferably 50 g/cm² to 400 g/cm², and more preferably 100 g/cm² to 300 g/cm².

In addition, the living body surface BS is preferably pressed along a direction orthogonal to the extending direction of the perforation P (see FIG. 18B). The phrase “pressed along a direction orthogonal to the extending direction of the perforation” is not limited to pressing only in the direction orthogonal to the extending direction of the perforation, and also includes pressing in a direction inclined at an angle equal to or less than a predetermined angle (for example, 30 degrees or less) with respect to the direction orthogonal to the extending direction of the perforation. The attachment device 1 of the present embodiment can press the living body surface BS along a direction orthogonal to the extending direction of the perforation P (see FIG. 18B).

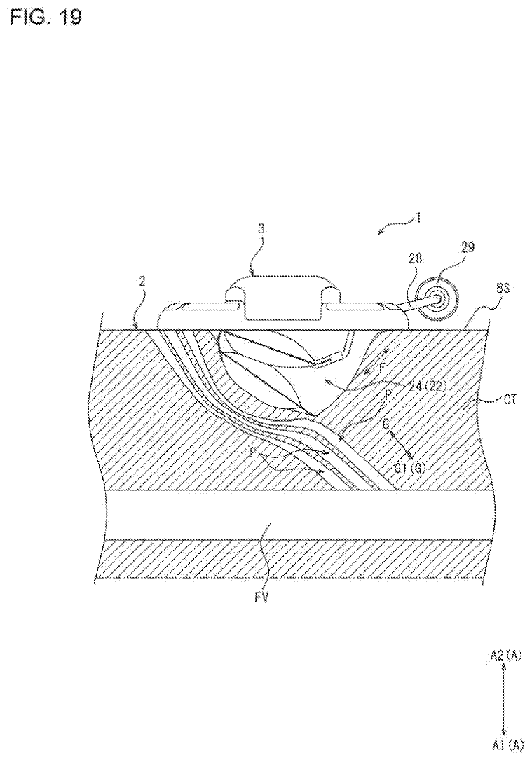

Specifically, as shown in FIG. 11, the expansion portion 24 of the present embodiment can be expanded in a direction inclined with respect to the thickness direction A. In this way, the living body surface can be pressed along a direction orthogonal to the extending direction of the perforation P (see FIG. 18B). Specifically, as shown in FIGS. 18A and 18B, a sheath as the insertion member 100 for medical use is inserted not in a direction (the same direction as the thickness direction A) orthogonal to the living body surface BS but in a direction inclined to one side with respect to the direction orthogonal to the living body surface BS. Therefore, as shown in FIG. 18B, the extending direction of the perforation P is also inclined with respect to the direction orthogonal to the living body surface BS. Therefore, in a case where the expansion portion 24 is expandable in a direction (hereinafter, may be referred to as “inclined direction F”) inclined to the opposite side to the extending direction of the perforation P with respect to the thickness direction A that is the direction orthogonal to the living body surface BS, the living body surface BS can be easily pressed along the direction orthogonal to the extending direction of the perforation P. Therefore, it is easy to realize the attachment device 1 that narrows or occludes the perforation P without occluding the vein such as the femoral vein FV in FIGS. 18A and 18B. FIG. 19 is a view showing a state in which the attachment device 1 presses the living body surface BS in the inclined direction F to narrow or occlude the perforation P shown in FIG. 18B. As shown in FIG. 19, according to the attachment device 1, the perforation P is more easily narrowed or occluded without further occluding the vein such as the femoral vein FV.

According to the living body pressing method shown in FIGS. 12A to 12E, 13, and 14A to 14G, hemostasis can be achieved by narrowing or occluding the perforation P (see FIG. 18B) without occluding the vein such as the femoral vein FV. In particular, by realizing the above-described living body pressing method with the attachment device 1, it is possible to achieve hemostasis by a simple method without the need for pressing by a medical worker's hand, using a large-scale hemostatic device, or the like.

### Pressing on Living Body Surface by Attachment Device 1

As shown in FIG. 19, in the attachment device 1, in a state in which the attachment sheet 2 is mounted on a living body, the expansion portion 24 can press a living body surface in the inclined direction F inclined with respect to a vertical direction (that is the same direction as the thickness direction A in FIG. 19, and is the vertical direction in FIG. 19. Hereinafter, it is simply referred to as “vertical direction”) perpendicular to the living body surface BS. In this way, as shown in FIG. 19, the perforation P is easily narrowed or occluded without occluding the vein such as the femoral vein FV.

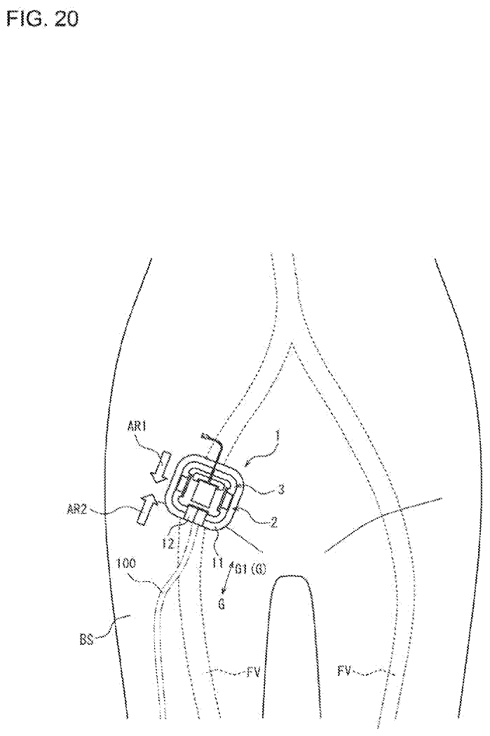

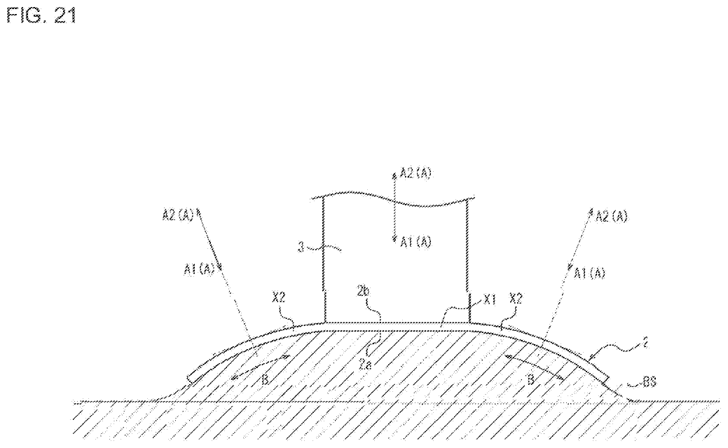

FIG. 20 is a front view of the state shown in FIG. 19 as viewed from the living body surface BS side. In other words, FIG. 20 shows a front view of the living body surface BS at a position pressed by the attachment device 1. Here, the phrase “front view of the living body surface at position pressed by the attachment device” means a state in which a part of the living body surface to be pressed by the attachment device is viewed from a direction perpendicular to the part before pressing. FIG. 20 shows a front view of the groin. In the front view shown in FIG. 20, the direction (see outlined arrow “AR1” in FIG. 20) in which the living body surface BS is pressed faces a sheath insertion direction G1 (see outlined arrow “AR2” in FIG. 20) from the living body surface BS toward the vein in an extending direction G of the perforation P. That is, the direction in which the attachment device 1 presses the living body surface BS faces the sheath insertion direction G1 in the front view shown in FIG. 20. In this way, the perforation P (see FIGS. 18B and FIG. 19) is easily narrowed or occluded without occluding the vein such as the femoral vein FV.

In other words, as shown in FIG. 19, the extending direction G of the perforation P is inclined with respect to the living body surface BS and is also inclined with respect to the vertical direction (the vertical direction in FIG. 19) perpendicular to the living body surface BS. In addition, as shown in FIG. 19, the direction in which the living body surface BS is pressed by the attachment device 1 is inclined with respect to the living body surface BS, and is also inclined with respect to the vertical direction (the vertical direction in FIG. 19) perpendicular to the living body surface BS. Furthermore, as shown in FIG. 19, the extending direction G of the perforation P is inclined to the opposite side to the inclined direction F as the direction in which the living body surface is pressed by the attachment device 1, with respect to the vertical direction (the vertical direction in FIG. 19). That is, the living body surface is pressed by the attachment device 1 so that the pressing direction intersects with the extending direction G of the perforation P. As a result, the perforation P can be efficiently narrowed or occluded.

### Second Embodiment

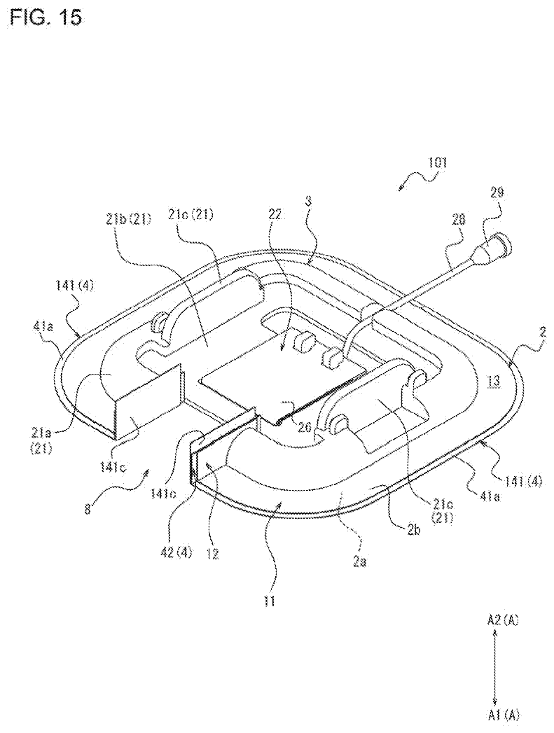

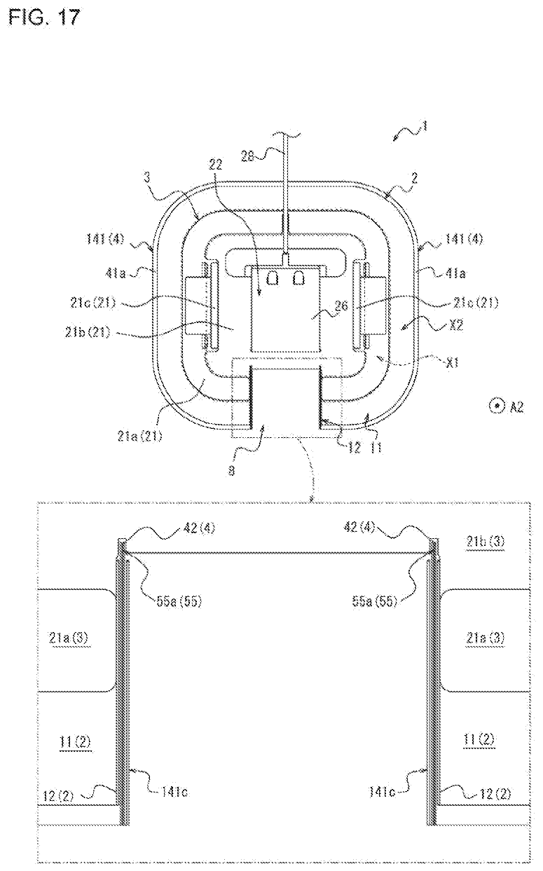

Next, an attachment device 101 as a second embodiment of the present disclosure will be described with reference to FIGS. 15 to 17. FIG. 15 is a perspective view of the attachment device 101 from above. FIGS. 16A to 16E are explanatory views explaining a procedure for removing a first release sheet 141 and a second release sheet 42 of the attachment device 101. The view on the upper side in FIG. 17 is a top view of the attachment device 101, and the view on the lower side in FIG. 17 is an enlarged view of a part of the top view of the attachment device 101.

The attachment device 101 of the present embodiment is different from the attachment device 1 (see, for example, FIG. 1) of the first embodiment described above mainly in the length of the flap portion 12 of the attachment sheet 2, the presence or absence of a locking portion 55 of the device body 3, and the shape of the first release sheet 141. Here, only the differences will be described, and description of other common configurations will be omitted.

As shown in FIG. 16E, flap portions 12 of the present embodiment cover only a part of a receiving portion 8. With such a configuration, the flap portions 12 provided to extend from both ends of a sheet body 11 extending in a substantially C-shape in plan view (see FIG. 17) do not interfere with each other. Therefore, it is possible to suppress unintended sticking of attachment surfaces of the flap portions 12 to each other.

In addition, as shown in FIG. 17, the device body 3 of the present embodiment includes the locking portion 55 that locks the second release sheet 42 in a state in which an outer edge portion 13 of the attachment sheet 2 is bent toward the fixing surface 2b side. More specifically, the locking portion 55 of the present embodiment locks the second release sheet 42 attached to the flap portion 12 in a state bent upward with respect to the sheet body 11. Therefore, the flap portion 12 and the second release sheet 42 attached to the flap portion 12 can be maintained in a posture standing upward with respect to the sheet body 11.

The locking portion 55 may lock, in addition to or instead of the second release sheet 42, an overlapping portion 141c of the first release sheet 141 to be described later. However, the locking portion 55 preferably locks at least the second release sheet 42 as in the present embodiment. By locking the second release sheet 42 by the locking portion 55, regardless of whether before or after removal of the first release sheet 141 including the overlapping portion 141c to be described later from an attachment surface 2a, the flap portion 12 and the second release sheet 42 attached to the flap portion 12 can be reliably maintained in a posture standing upward with respect to the sheet body 11.

Further, particularly preferably, the locking portion 55 locks the second release sheet 42 and does not lock the overlapping portion 141c of the first release sheet 141. Since the locking portion 55 does not lock the overlapping portion 141c of the first release sheet 141, it is possible to suppress a deterioration in operability in removal of the first release sheet 141 from the attachment surface 2a.

In the present embodiment, a cover portion 21b of a holding body 21 of the device body 3 includes the locking portion 55. Specifically, the locking portion 55 of the present embodiment is a recess 55a formed on an end surface of the cover portion 21b facing the receiving portion 8. An outer edge portion of the second release sheet 42 attached to the flap portion 12 in a state bent upward with respect to the sheet body 11 enters the recess 55a. As a result, it is possible to suppress falling of the flap portion 12 and the second release sheet 42 attached to the flap portion 12, and thus an operation of receiving an insertion member 100 (see, for example, FIG. 14B) in the receiving portion 8 is easily performed. The locking portion 55 of the present embodiment is the recess 55a described above, but the configuration of the locking portion 55 is not limited to the configuration of the present embodiment.

Next, the configuration of the first release sheet 141 of the present embodiment will be described. The first release sheet 141 of the present embodiment includes a first release layer 41a, a first folding layer 41b, and the overlapping portion 141c. The first folding layer 41b of the present embodiment consists only of one layer.

The overlapping portion 141c of the present embodiment extends along a second attachment region (the attachment surface of the flap portion 12 in the present embodiment) and covers the second release sheet 42. More specifically, the overlapping portion 141c of the present embodiment is formed of parts provided to extend respectively from the first release layer 41a and the first folding layer 41b, and a second fold 30b. The overlapping portion 141c is bent in an upward direction A2 and provided to extend from the first release layer 41a and the first folding layer 41b. By providing such an overlapping portion 141c, it is possible to suppress falling of the flap portion 12 and the second release sheet 42 attached to the flap portion 12, and thus an operation of receiving an insertion member 100 (see, for example, FIG. 14B) in the receiving portion 8 is easily performed. In the present embodiment, the overlapping portion 141c is provided in addition to the locking portion 55 described above, but only one of them may be provided.

The attachment device and the attachment method according to the present disclosure are not limited to the specific configurations and steps shown in the above-described embodiments, and various modifications, changes, and combinations are possible without departing from the scope of the claims. The attachment device 1 of the first embodiment described above may further include, for example, the locking portion 55 of the attachment device 101 of the second embodiment described above.

The detailed description above describes to an attachment device and an attachment method. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. An attachment device comprising:
an attachment sheet including an attachment surface configured to be attached to a living body surface and a fixing surface that is positioned on a side opposite to the attachment surface;
a device body that is fixed to the fixing surface of the attachment sheet;
a plurality of release sheets that are releasably attached to different positions on the attachment surface of the attachment sheet;
the attachment sheet includes an outer edge portion to which the device body is not fixed, the outer edge portion is positioned at an outer periphery of the device body in plan view as viewed in a thickness direction of the attachment sheet; and
wherein the plurality of release sheets include a first release sheet that is attached to a first attachment region including an attachment surface of a part different from the outer edge portion in the attachment surface of the attachment sheet, and a second release sheet that is attached to a second attachment region including only an attachment surface of the outer edge portion in the attachment surface of the attachment sheet.

2. The attachment device according to claim 1, wherein
the first release sheet includes a first release layer that is attached to the first attachment region of the attachment surface of the attachment sheet; and
a first folding layer that is folded and laminated on the first release layer on a side of the attachment surface opposite to a first attachment region side.

3. The attachment device according to claim 2, wherein
the second release sheet includes a second release layer that is attached to the second attachment region of the attachment surface of the attachment sheet; and
a second folding layer that is folded and laminated on the second release layer on a side of the attachment surface opposite to a second attachment region side.

4. The attachment device according to claim 3, wherein a first folding direction of the first release sheet from the first release layer to the first folding layer is different from a second folding direction of the second release sheet from the second release layer to the second folding layer.

5. The attachment device according to claim 4, wherein the first folding direction and the second folding direction are in mutually opposite directions in an in-plane direction of the attachment surface of the attachment sheet.

6. The attachment device according to claim 1, wherein the second attachment region is adjacent to the first attachment region.

7. The attachment device according to claim 6, wherein the first release sheet includes an overlapping portion that covers the second release sheet.

8. The attachment device according to claim 7, wherein
a boundary between the first attachment region and the second attachment region is defined by a ridgeline formed by bending of the outer edge portion of the attachment sheet toward a fixing surface side; and
the overlapping portion of the first release sheet protrudes in a direction away from the second release sheet along the first attachment region, not along the second attachment region.

9. The attachment device according to claim 7, wherein
a boundary between the first attachment region and the second attachment region is defined by a ridgeline formed by bending of the outer edge portion of the attachment sheet toward a fixing surface side; and
the overlapping portion of the first release sheet extends along the second attachment region and covers the second release sheet.

10. The attachment device according to claim 1, wherein the device body includes a locking portion that locks the second release sheet in a state in which the outer edge portion of the attachment sheet is bent toward the fixing surface side.

11. The attachment device according to claim 10, wherein the locking portion is a recess that an outer edge portion of the second release sheet enters.

12. An attachment device comprising:
an attachment sheet including an attachment surface configured to be attached to a living body surface and a fixing surface that is positioned on a side opposite to the attachment surface, the fixing surface configured to receive a device body including a pressing portion configured to press the living body surface in a central region of the attachment sheet;
a plurality of release sheets that are releasably attached to different positions on the attachment surface of the attachment sheet; and
wherein the plurality of release sheets include a first release sheet that is attached to a first attachment region including an attachment surface of a part different from an outer edge portion in the attachment surface of the attachment sheet, and a second release sheet that is attached to a second attachment region including only an attachment surface of the outer edge portion in the attachment surface of the attachment sheet.

13. The attachment device according to claim 12, wherein the outer edge portion corresponds to an outer periphery of the device body.

14. The attachment device according to claim 12, wherein
the first release sheet includes a first release layer that is attached to the first attachment region of the attachment surface of the attachment sheet;
a first folding layer that is folded and laminated on the first release layer on a side of the attachment surface opposite to a first attachment region side;
the second release sheet includes a second release layer that is attached to the second attachment region of the attachment surface of the attachment sheet; and
a second folding layer that is folded and laminated on the second release layer on a side of the attachment surface opposite to a second attachment region side.

15. The attachment device according to claim 14, wherein
a first folding direction of the first release sheet from the first release layer to the first folding layer is different from a second folding direction of the second release sheet from the second release layer to the second folding layer; and
the first folding direction and the second folding direction are in mutually opposite directions in an in-plane direction of the attachment surface of the attachment sheet.

16. The attachment device according to claim 12, wherein the second attachment region is adjacent to the first attachment region, and the first release sheet includes an overlapping portion that covers the second release sheet.

17. The attachment device according to claim 16, wherein
a boundary between the first attachment region and the second attachment region is defined by a ridgeline formed by bending of the outer edge portion of the attachment sheet toward a fixing surface side; and
the overlapping portion of the first release sheet protrudes in a direction away from the second release sheet along the first attachment region, not along the second attachment region.

18. The attachment device according to claim 16, wherein
a boundary between the first attachment region and the second attachment region is defined by a ridgeline formed by bending of the outer edge portion of the attachment sheet toward a fixing surface side; and
the overlapping portion of the first release sheet extends along the second attachment region and covers the second release sheet.

19. The attachment device according to claim 12, further comprising:
the device body, and wherein the device body includes a locking portion that locks the second release sheet in a state in which the outer edge portion of the attachment sheet is bent toward the fixing surface side, and the locking portion is a recess that an outer edge portion of the second release sheet enters.

20. An attachment method for attachment to a living body surface by an attachment device, the attachment device including an attachment sheet including an attachment surface that is attachable to a living body surface and a fixing surface that is positioned on a side opposite to the attachment surface, a device body that is fixed to the fixing surface of the attachment sheet, and first and second release sheets that are releasably attached to different positions on the attachment surface of the attachment sheet, the first release sheet being attached to a first attachment region in the attachment surface of the attachment sheet, and the second release sheet being attached to a second attachment region in the attachment surface of the attachment sheet, the method comprising:
releasing the first release sheet from the first attachment region, and attaching the first attachment region to the living body surface; and
releasing the second release sheet from the second attachment region by moving the second release sheet in a direction orthogonal to the first attachment region in a state attached to the living body surface, and attaching the second attachment region to the living body surface.
